SEQUENCE LISTING (166 sequences)

SEQ ID NO: 1 - DNA, 63 bp, Artificial Sequence
  Synthetic construct
tcgtcggcagcgtcagatgtgtataagagacagccttatattcccagggccggttcgcgatcg

SEQ ID NO: 2 - DNA, 64 bp, Artificial Sequence
  Synthetic construct
gtctcgtgggctcggagatgtgtataagagacaggccgccctcgaccgccttgattctcatggt

SEQ ID NO: 3 - DNA, 60 bp, Artificial Sequence
  Synthetic construct
  misc_feature (15)..(19): n is a, c, g, t or u
cagtaccctgcaggnnnnntagttattaatgactcacggggatttccaagtctccacccc

SEQ ID NO: 4 - DNA, 44 bp, Artificial Sequence
  Synthetic construct
cagtacggcgcgcccaatttacgcgttaagatacattgatgagt

SEQ ID NO: 5 - DNA, 36 bp, Artificial Sequence
  Synthetic construct
cagtacggtaccaaggtcgggcaggaagagggccta

SEQ ID NO: 6 - DNA, 87 bp, Artificial Sequence
  Synthetic construct
cggactagccttattttaacttgctatttctagctctaaaacccctcgaccgccttgattccggtgtttcgtcctttccacaagata

SEQ ID NO: 7 - DNA, 76 bp, Artificial Sequence
  Synthetic construct
cagtactctagaaaaaaagcaccgactcggtgccactttttcaagttgataacggactagccttattttaacttgc

SEQ ID NO: 8 - DNA, 87 bp, Artificial Sequence
  Synthetic construct
cggactagccttattttaacttgctatttctagctctaaaaccggatgtgcacttgaagtgcggtgtttcgtcctttccacaagata

SEQ ID NO: 9 - DNA, 87 bp, Artificial Sequence
  Synthetic construct
cggactagccttattttaacttgctatttctagctctaaaactcggatgtgcacttgaagtcggtgtttcgtcctttccacaagata

SEQ ID NO: 10 - DNA, 87 bp, Artificial Sequence
  Synthetic construct
cggactagccttattttaacttgctatttctagctctaaaactcgtagggcttgccttcgccggtgtttcgtcctttccacaagata

SEQ ID NO: 11 - DNA, 87 bp, Artificial Sequence
  Synthetic construct
cggactagccttattttaacttgctatttctagctctaaaactcgccctcggatgtgcactcggtgtttcgtcctttccacaagata

SEQ ID NO: 12 - DNA, 33 bp, Artificial Sequence
  Synthetic construct
tcgtcggcagcgtcagatgtgtataagagacag

SEQ ID NO: 13 - DNA, 34 bp, Artificial Sequence
  Synthetic construct
gtctcgtgggctcggagatgtgtataagagacag

SEQ ID NO: 14 - DNA, 19 bp, Artificial Sequence
  Synthetic construct
ttcaagtgcacatccgagg

SEQ ID NO: 15 - DNA, 34 bp, Artificial Sequence
  Synthetic construct
ttcaagtgcacatccgaaggcaagccctacgagg

SEQ ID NO: 16 - DNA, 19 bp, Artificial Sequence
  Synthetic construct
cctcggatgtgcacttgaa

SEQ ID NO: 17 - DNA, 8601 bp, Artificial Sequence
  Synthetic construct
  misc_feature (1015)..(1019): n is a, c, g, t or u
taggggttccgcgcacatttccccgaaaagtgccacctggccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttcgcgatcgccctgcaggnnnnntagttattaatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgaccagctaagacactgccacggtcagatccgctagcgctaccggtcgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagaggtggaggaggttccggatctcacggcttccctcccgaggtggaggagcaggccgccggcaccctgcccatgagctgcgcccaggagagcggcatggatagacaccctgctgcttgcgccagcgccaggatcaacgtctctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgggcgcgcccttaagctgggacggaggcttgtttgcgaggccgcggccggccgaagttcctattctctagaaagtataggaacttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgatcgagcagctgaagcttaccgcaggctatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggggaggctaactgaagcttcccgggggtaccaaattcgtcgacagatctaacttgtttattgcagcttataatggttacaaataaagcaatagcatcacaaatttcacaaataaagcatttttttcactgcattctagttgtggtttgtccaaactcatcaatgtatcttatgatgtctgcatatggaagttcctattctctagaaagtataggaacttcgcggccgctcccacccgctcgtccccccgcgcacctttgctaggagcgggtcgcccatgtggctctcaggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcaccggtacccggccgcgactctagatcataatcagctcgagccttaacaagcttcgaaacgatatgggctgaatacaaaaacgatatgggctgaatacaaaaacgatatgggctgaatacaaaccgcttgaagtctttaattaaaccgcttgaagtctttaattaaaccgcttgaagtctttaattaaaggatccaccggatctagataactgatcataatcgcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaa

SEQ ID NO: 18 - DNA, 962 bp, Artificial Sequence
  Synthetic construct
  misc_feature (958)..(963): N is any A, T, C, or G nucleotide
ccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttnnnnn

SEQ ID NO: 19 - DNA, 225 bp, Artificial Sequence
  Synthetic construct
gactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgaccagctaagacactgccacggtcagatccgctagcgctaccggtcgccacc

SEQ ID NO: 20 - DNA, 696 bp, Artificial Sequence
  Synthetic construct
atggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaga

SEQ ID NO: 21 - DNA, 625 bp, Artificial Sequence
  Synthetic construct
ctgggacggaggcttgtttgcgaggccgcggccggccgaagttcctattctctagaaagtataggaacttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgatcgagcagctgaagcttaccgcaggct

SEQ ID NO: 22 - DNA, 1023 bp, Artificial Sequence
  Synthetic construct
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaa

SEQ ID NO: 23 - DNA, 1065 bp, Artificial Sequence
  Synthetic construct
ggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcac

SEQ ID NO: 24 - DNA, 17 bp, Artificial Sequence
  Synthetic construct
aggcaagccctacgagg

SEQ ID NO: 25 - DNA, 34 bp, Artificial Sequence; identical to SEQ ID NO: 15
  Synthetic construct

SEQ ID NO: 26 - DNA, 24 bp, Artificial Sequence
  Synthetic construct
ttcaagtgcacatccgaggggagg

SEQ ID NO: 27 - DNA, 38 bp, Artificial Sequence
  Synthetic construct
ttcaagtgcacatccgagcgaaggcaagccctacgagg

SEQ ID NO: 28 - DNA, 19 bp, Artificial Sequence
  Synthetic construct
ttcaagtgcacatccgagg

SEQ ID NO: 29 - DNA, 34 bp, Artificial Sequence
  Synthetic construct
ttcaagtgcacatccgagggcaagccctacgagg

SEQ ID NO: 30 - DNA, 39 bp, Artificial Sequence
  Synthetic construct
ttcaagtgcacatccgaggggaaggcaagccctacgagg

SEQ ID NO: 31 - DNA, 33 bp, Artificial Sequence
  Synthetic construct
ttcaagtgcacatccgaggcaagccctacgagg

SEQ ID NO: 32 - DNA, 33 bp, Artificial Sequence
  Synthetic construct
ttcaagtgcacatcgaaggcaagccctacgagg

SEQ ID NO: 33 - DNA, 38 bp, Artificial Sequence
  Synthetic construct
ttcaagtgcacatccgagggaaggcaagccctacgagg

SEQ ID NO: 34 - DNA, 36 bp, Artificial Sequence
  Synthetic construct
ttcaagtgcacatccgcgaaggcaagccctacgagg

SEQ ID NO: 35 - DNA, 19 bp, Artificial Sequence
  Synthetic construct
ttcaagtgcacatccgagg

SEQ ID NO: 36 - DNA, 34 bp, Artificial Sequence; identical to SEQ ID NO: 15
  Synthetic construct

SEQ ID NO: 37 - DNA, 34 bp, Artificial Sequence; identical to SEQ ID NO: 29
  Synthetic construct

SEQ ID NO: 38 - DNA, 38 bp, Artificial Sequence; identical to SEQ ID NO: 27
  Synthetic construct

SEQ ID NO: 39 - DNA, 5 bp, Artificial Sequence
  Synthetic construct
ttcaa

SEQ ID NO: 40 - DNA, 38 bp, Artificial Sequence; identical to SEQ ID NO: 27
  Synthetic construct

SEQ ID NO: 41 - DNA, 34 bp, Artificial Sequence; identical to SEQ ID NO: 15
  Synthetic construct

SEQ ID NO: 42 - DNA, 39 bp, Artificial Sequence; identical to SEQ ID NO: 30
  Synthetic construct

SEQ ID NO: 43 - DNA, 39 bp, Artificial Sequence
  Synthetic construct
ttcaagtgcacatccgaggcgaaggcaagccctacgagg

SEQ ID NO: 44 - DNA, 34 bp, Artificial Sequence; identical to SEQ ID NO: 29
  Synthetic construct

SEQ ID NO: 45 - DNA, 38 bp, Artificial Sequence; identical to SEQ ID NO: 27
  Synthetic construct

SEQ ID NO: 46 - DNA, 39 bp, Artificial Sequence; identical to SEQ ID NO: 30
  Synthetic construct

SEQ ID NO: 47 - DNA, 34 bp, Artificial Sequence; identical to SEQ ID NO: 15
  Synthetic construct

SEQ ID NO: 48 - DNA, 39 bp, Artificial Sequence; identical to SEQ ID NO: 43
  Synthetic construct

SEQ ID NO: 49 - DNA, 34 bp, Artificial Sequence; identical to SEQ ID NO: 29
  Synthetic construct

SEQ ID NO: 50 - DNA, 34 bp, Artificial Sequence; identical to SEQ ID NO: 15
  Synthetic construct

SEQ ID NO: 51 - DNA, 19 bp, Artificial Sequence
  Synthetic construct
ttcaagtgcacatccgagg

SEQ ID NO: 52 - DNA, 34 bp, Artificial Sequence; identical to SEQ ID NO: 29
  Synthetic construct

SEQ ID NO: 53 - DNA, 38 bp, Artificial Sequence; identical to SEQ ID NO: 27
  Synthetic construct

SEQ ID NO: 54 - DNA, 39 bp, Artificial Sequence; identical to SEQ ID NO: 43
  Synthetic construct

SEQ ID NO: 55 - DNA, 38 bp, Artificial Sequence; identical to SEQ ID NO: 27
  Synthetic construct

SEQ ID NO: 56 - DNA, 39 bp, Artificial Sequence; identical to SEQ ID NO: 30
  Synthetic construct

SEQ ID NO: 57 - DNA, 34 bp, Artificial Sequence; identical to SEQ ID NO: 15
  Synthetic construct

SEQ ID NO: 58 - DNA, 39 bp, Artificial Sequence; identical to SEQ ID NO: 43
  Synthetic construct

SEQ ID NO: 59 - DNA, 34 bp, Artificial Sequence; identical to SEQ ID NO: 29
  Synthetic construct

SEQ ID NO: 60 - DNA, 38 bp, Artificial Sequence; identical to SEQ ID NO: 27
  Synthetic construct

SEQ ID NO: 61 - DNA, 34 bp, Artificial Sequence; identical to SEQ ID NO: 15
  Synthetic construct

SEQ ID NO: 62 - DNA, 39 bp, Artificial Sequence; identical to SEQ ID NO: 30
  Synthetic construct

SEQ ID NO: 63 - DNA, 39 bp, Artificial Sequence; identical to SEQ ID NO: 43
  Synthetic construct

SEQ ID NO: 64 - DNA, 34 bp, Artificial Sequence; identical to SEQ ID NO: 29
  Synthetic construct

SEQ ID NO: 65 - DNA, 38 bp, Artificial Sequence
  Synthetic construct
cctcgtagggcttgccttcgctcggatgtgcacttgaa

SEQ ID NO: 66 - DNA, 39 bp, Artificial Sequence
  Synthetic construct
cctcgtagggcttgccttcccctcggatgtgcacttgaa

SEQ ID NO: 67 - DNA, 34 bp, Artificial Sequence
  Synthetic construct
cctcgtagggcttgccttcggatgtgcacttgaa

SEQ ID NO: 68 - DNA, 34 bp, Artificial Sequence
  Synthetic construct
cctcgtagggcttgccctcggatgtgcacttgaa

SEQ ID NO: 69 - DNA, 39 bp, Artificial Sequence
  Synthetic construct
cctcgtagggcttgccttcgcctcggatgtgcacttgaa

SEQ ID NO: 70 - DNA, 38 bp, Artificial Sequence; identical to SEQ ID NO: 65
  Synthetic construct

SEQ ID NO: 71 - DNA, 39 bp, Artificial Sequence; identical to SEQ ID NO: 66
  Synthetic construct

SEQ ID NO: 72 - DNA, 34 bp, Artificial Sequence; identical to SEQ ID NO: 67
  Synthetic construct

SEQ ID NO: 73 - DNA, 39 bp, Artificial Sequence; identical to SEQ ID NO: 69
  Synthetic construct

SEQ ID NO: 74 - DNA, 34 bp, Artificial Sequence; identical to SEQ ID NO: 68
  Synthetic construct

SEQ ID NO: 75 - DNA, 39 bp, Artificial Sequence; identical to SEQ ID NO: 69
  Synthetic construct

SEQ ID NO: 76 - DNA, 39 bp, Artificial Sequence; identical to SEQ ID NO: 66
  Synthetic construct

SEQ ID NO: 77 - DNA, 19 bp, Artificial Sequence
  Synthetic construct
cctcggatgtgcacttgaa

SEQ ID NO: 78 - DNA, 38 bp, Artificial Sequence; identical to SEQ ID NO: 65
  Synthetic construct

SEQ ID NO: 79 - DNA, 34 bp, Artificial Sequence; identical to SEQ ID NO: 67
  Synthetic construct

SEQ ID NO: 80 - DNA, 38 bp, Artificial Sequence; identical to SEQ ID NO: 65
  Synthetic construct

SEQ ID NO: 81 - DNA, 34 bp, Artificial Sequence; identical to SEQ ID NO: 68
  Synthetic construct

SEQ ID NO: 82 - DNA, 39 bp, Artificial Sequence; identical to SEQ ID NO: 66
  Synthetic construct

SEQ ID NO: 83 - DNA, 23 bp, Artificial Sequence
  Synthetic construct
cctcgtagggcttgccttcggaa

SEQ ID NO: 84 - DNA, 19 bp, Artificial Sequence
  Synthetic construct
cctcggatgtgcacttgaa

SEQ ID NO: 85 - DNA, 19 bp, Artificial Sequence
  Synthetic construct
ttcaagtgcacatccgagg

SEQ ID NO: 86 - DNA, 34 bp, Artificial Sequence; identical to SEQ ID NO: 15
  Synthetic construct

SEQ ID NO: 87 - DNA, 34 bp, Artificial Sequence; identical to SEQ ID NO: 29
  Synthetic construct

SEQ ID NO: 88 - DNA, 38 bp, Artificial Sequence; identical to SEQ ID NO: 27
  Synthetic construct

SEQ ID NO: 89 - DNA, 39 bp, Artificial Sequence; identical to SEQ ID NO: 43
  Synthetic construct

SEQ ID NO: 90 - DNA, 24 bp, Artificial Sequence
  Synthetic construct
atcggttcaagtgcacatccgagg

SEQ ID NO: 91 - DNA, 39 bp, Artificial Sequence
  Synthetic construct
atcggttcaagtgcacatccgaaggcaagccctacgagg

SEQ ID NO: 92 - DNA, 39 bp, Artificial Sequence
  Synthetic construct
aatcggttcaagtgcacatccgagggcaagccctacgag

SEQ ID NO: 93 - DNA, 43 bp, Artificial Sequence
  Synthetic construct
atcggttcaagtgcacatccgagcgaaggcaagccctacgagg

SEQ ID NO: 94 - DNA, 44 bp, Artificial Sequence
  Synthetic construct
atcggttcaagtgcacatccgaggcgaaggcaagccctacgagg

SEQ ID NO: 95 - DNA, 24 bp, Artificial Sequence
  Synthetic construct
aaaaattcaagtgcacatccgagg

SEQ ID NO: 96 - DNA, 39 bp, Artificial Sequence
  Synthetic construct
aaaaattcaagtgcacatccgaaggcaagccctacgagg

SEQ ID NO: 97 - DNA, 24 bp, Artificial Sequence
  Synthetic construct
aatggttcaagtgcacatccgagg

SEQ ID NO: 98 - DNA, 39 bp, Artificial Sequence
  Synthetic construct
aaaaattcaagtgcacatccgagggcaagccctacgagg

SEQ ID NO: 99 - DNA, 43 bp, Artificial Sequence
  Synthetic construct
aaaaattcaagtgcacatccgagcgaaggcaagccctacgagg

SEQ ID NO: 100 - DNA, 44 bp, Artificial Sequence
  Synthetic construct
aaaaattcaagtgcacatccgaggcgaaggcaagccctacgagg

SEQ ID NO: 101 - DNA, 39 bp, Artificial Sequence
  Synthetic construct
aatggttcaagtgcacatccgaaggcaagccctacgagg

SEQ ID NO: 102 - DNA, 19 bp, Artificial Sequence
  Synthetic construct
gaatcaaggcggtcgaggg

SEQ ID NO: 103 - DNA, 19 bp, Artificial Sequence
  Synthetic construct
cacttcaagtgcacatccg

SEQ ID NO: 104 - DNA, 19 bp, Artificial Sequence
  Synthetic construct
acttcaagtgcacatccga

SEQ ID NO: 105 - DNA, 19 bp, Artificial Sequence
  Synthetic construct
gcgaaggcaagccctacga

SEQ ID NO: 106 - DNA, 19 bp, Artificial Sequence
  Synthetic construct
agtgcacatccgagggcga

SEQ ID NO: 107 - DNA, 38 bp, Artificial Sequence; identical to SEQ ID NO: 65
  Synthetic construct

SEQ ID NO: 108 - DNA, 39 bp, Artificial Sequence; identical to SEQ ID NO: 66
  Synthetic construct

SEQ ID NO: 109 - DNA, 34 bp, Artificial Sequence; identical to SEQ ID NO: 67
  Synthetic construct

SEQ ID NO: 110 - DNA, 34 bp, Artificial Sequence; identical to SEQ ID NO: 68
  Synthetic construct

SEQ ID NO: 111 - DNA, 39 bp, Artificial Sequence; identical to SEQ ID NO: 69
  Synthetic construct

SEQ ID NO: 112 - DNA, 38 bp, Artificial Sequence; identical to SEQ ID NO: 65
  Synthetic construct

SEQ ID NO: 113 - DNA, 39 bp, Artificial Sequence; identical to SEQ ID NO: 66
  Synthetic construct

SEQ ID NO: 114 - DNA, 34 bp, Artificial Sequence; identical to SEQ ID NO: 67
  Synthetic construct

SEQ ID NO: 115 - DNA, 34 bp, Artificial Sequence; identical to SEQ ID NO: 68
  Synthetic construct

SEQ ID NO: 116 - DNA, 39 bp, Artificial Sequence; identical to SEQ ID NO: 69
  Synthetic construct

SEQ ID NO: 117 - DNA, 38 bp, Artificial Sequence; identical to SEQ ID NO: 65
  Synthetic construct

SEQ ID NO: 118 - DNA, 39 bp, Artificial Sequence; identical to SEQ ID NO: 66
  Synthetic construct

SEQ ID NO: 119 - DNA, 39 bp, Artificial Sequence; identical to SEQ ID NO: 69
  Synthetic construct

SEQ ID NO: 120 - DNA, 34 bp, Artificial Sequence; identical to SEQ ID NO: 67
  Synthetic construct

SEQ ID NO: 121 - DNA, 34 bp, Artificial Sequence; identical to SEQ ID NO: 68
  Synthetic construct

SEQ ID NO: 122 - DNA, 38 bp, Artificial Sequence; identical to SEQ ID NO: 65
  Synthetic construct

SEQ ID NO: 123 - DNA, 33 bp, Artificial Sequence
  Synthetic construct
cctcgtagggcttgccttcggatgtcacttgaa

SEQ ID NO: 124 - DNA, 39 bp, Artificial Sequence; identical to SEQ ID NO: 66
  Synthetic construct

SEQ ID NO: 125 - DNA, 39 bp, Artificial Sequence; identical to SEQ ID NO: 69
  Synthetic construct

SEQ ID NO: 126 - DNA, 34 bp, Artificial Sequence; identical to SEQ ID NO: 68
  Synthetic construct

SEQ ID NO: 127 - DNA, 38 bp, Artificial Sequence; identical to SEQ ID NO: 65
  Synthetic construct

SEQ ID NO: 128 - DNA, 39 bp, Artificial Sequence; identical to SEQ ID NO: 66
  Synthetic construct

SEQ ID NO: 129 - DNA, 34 bp, Artificial Sequence; identical to SEQ ID NO: 67
  Synthetic construct

SEQ ID NO: 130 - DNA, 39 bp, Artificial Sequence; identical to SEQ ID NO: 69
  Synthetic construct

SEQ ID NO: 131 - DNA, 34 bp, Artificial Sequence; identical to SEQ ID NO: 68
  Synthetic construct

SEQ ID NO: 132 - DNA, 38 bp, Artificial Sequence; identical to SEQ ID NO: 65
  Synthetic construct

SEQ ID NO: 133 - DNA, 34 bp, Artificial Sequence; identical to SEQ ID NO: 67
  Synthetic construct

SEQ ID NO: 134 - DNA, 39 bp, Artificial Sequence; identical to SEQ ID NO: 66
  Synthetic construct

SEQ ID NO: 135 - DNA, 39 bp, Artificial Sequence; identical to SEQ ID NO: 69
  Synthetic construct

SEQ ID NO: 136 - DNA, 34 bp, Artificial Sequence; identical to SEQ ID NO: 68
  Synthetic construct

SEQ ID NO: 137 - DNA, 39 bp, Artificial Sequence; identical to SEQ ID NO: 69
  Synthetic construct

SEQ ID NO: 138 - DNA, 39 bp, Artificial Sequence; identical to SEQ ID NO: 69
  Synthetic construct

SEQ ID NO: 139 - DNA, 19 bp, Artificial Sequence
  Synthetic construct
cctcggatgtgcacttgaa

SEQ ID NO: 140 - DNA, 38 bp, Artificial Sequence; identical to SEQ ID NO: 65
  Synthetic construct

SEQ ID NO: 141 - DNA, 34 bp, Artificial Sequence; identical to SEQ ID NO: 67
  Synthetic construct

SEQ ID NO: 142 - DNA, 38 bp, Artificial Sequence; identical to SEQ ID NO: 65
  Synthetic construct

SEQ ID NO: 143 - DNA, 34 bp, Artificial Sequence; identical to SEQ ID NO: 68
  Synthetic construct

SEQ ID NO: 144 - DNA, 34 bp, Artificial Sequence; identical to SEQ ID NO: 68
  Synthetic construct

SEQ ID NO: 145 - DNA, 23 bp, Artificial Sequence
  Synthetic construct
cctcgtagggcttgccttcggaa

SEQ ID NO: 146 - DNA, 19 bp, Artificial Sequence
  Synthetic construct
cctcggatgtgcacttgaa

SEQ ID NO: 147 - DNA, 38 bp, Artificial Sequence; identical to SEQ ID NO: 65
  Synthetic construct

SEQ ID NO: 148 - DNA, 34 bp, Artificial Sequence; identical to SEQ ID NO: 67
  Synthetic construct

SEQ ID NO: 149 - DNA, 38 bp, Artificial Sequence
  Synthetic construct
cctcgtagggcttgccttccctcggatgtgcacttgaa

SEQ ID NO: 150 - DNA, 28 bp, Artificial Sequence
  Synthetic construct
cctcgtagggcttgccttcgcacttgaa

SEQ ID NO: 151 - DNA, 36 bp, Artificial Sequence
  Synthetic construct
cctcgtagggcttgccttcgcggatgtgcacttgaa

SEQ ID NO: 152 - DNA, 38 bp, Artificial Sequence; identical to SEQ ID NO: 65
  Synthetic construct

SEQ ID NO: 153 - DNA, 34 bp, Artificial Sequence; identical to SEQ ID NO: 67
  Synthetic construct

SEQ ID NO: 154 - DNA, 36 bp, Artificial Sequence; identical to SEQ ID NO: 151
  Synthetic construct

SEQ ID NO: 155 - DNA, 35 bp, Artificial Sequence
  Synthetic construct
cctcgtagggcttgccttcgggatgtgcacttgaa

SEQ ID NO: 156 - DNA, 39 bp, Artificial Sequence; identical to SEQ ID NO: 66
  Synthetic construct

SEQ ID NO: 157 - DNA, 38 bp, Artificial Sequence; identical to SEQ ID NO: 65
  Synthetic construct

SEQ ID NO: 158 - DNA, 34 bp, Artificial Sequence; identical to SEQ ID NO: 67
  Synthetic construct

SEQ ID NO: 159 - DNA, 31 bp, Artificial Sequence
  Synthetic construct
cctcgtagggcttgccttcggtgcacttgaa

SEQ ID NO: 160 - DNA, 19 bp, Artificial Sequence
  Synthetic construct
cctcggatgtgcacttgaa

SEQ ID NO: 161 - DNA, 30 bp, Artificial Sequence
  Synthetic construct
cctcgtagggcttgccttcgtgcacttgaa

SEQ ID NO: 162 - DNA, 33 bp, Artificial Sequence
  Synthetic construct
cctcgtagggcttgccttcgatctgcacttgaa

SEQ ID NO: 163 - DNA, 34 bp, Artificial Sequence
  Synthetic construct
cctcgtagggcttgccttcggatctgcacttgaa

SEQ ID NO: 164 - DNA, 28 bp, Artificial Sequence
  Synthetic construct
cctcgtagggcttgccttcgcacttgaa

SEQ ID NO: 165 - DNA, 34 bp, Artificial Sequence; identical to SEQ ID NO: 68
  Synthetic construct

SEQ ID NO: 166 - DNA, 23 bp, Artificial Sequence
  Synthetic construct
cctcgctcggatgtgcacttgaa

(19) United States
(12) Patent Application Publication
(10) Pub. No.: US 2023/0183749 A1
(43) Pub. Date: Jun. 15, 2023

(54) GENETIC PHYSICAL UNCLONABLE FUNCTIONS AND METHODS OF USE THEREOF

(71) Applicant: BOARD OF REGENTS, THE UNIVERSITY OF TEXAS SYSTEM, Austin, TX (US)

(72) Inventors: Leonidas BLERIS, Allen, TX (US); Georgios MAKRIS, Dallas, TX (US); Yi LI, Garland, TX (US)

(21) Appl. No.: 17/999,297

(22) PCT Filed: May 19, 2021
(86) PCT No.: PCT/US2021/033108

§ 371 Date: Nov. 18, 2022

Related U.S. Application Data
(60) Provisional application No. 63/027,331, filed on May 19, 2020.

(51) Int. Cl. C12N 15/90; C12N 15/113; C12N 15/85; C12N 9/22

(52) U.S. Cl. CPC C12N 15/907; C12N 15/113; C12N 15/85; C12N 9/22; C12N 2310/20

(57) ABSTRACT

The present disclosure relates to compositions, cells, and methods for authentication of cell lines using genetic physical unclonable functions.

### CROSS REFERENCE TO RELATED APPLICATIONS

This application claims the benefit of U.S. Provisional Patent Application Ser. No. 63/027,331 filed May 19, 2020, the disclosure of which is expressly incorporated herein by reference.

### FIELD

The present disclosure relates to compositions, cells, and methods for authentication of cell lines using genetic physical unclonable functions.

### BACKGROUND

Recent advances in synthetic biology and genome editing have enabled development of a broad range of engineered cells and have fueled emergence of a novel industry which seeks to produce specialized cell lines and monetize them through commercial distribution networks. Many such highly customized proprietary cell lines are the result of extensive and expensive research and development efforts and come with price tags in the tens of thousands of dollars. Therefore, the legitimate producers of these valuable cell lines have a vested interest to protect their intellectual property and recover their investment by ensuring that their proprietary cell line does not get illicitly copied and distributed. At the same time, customers who acquire such expensive cell lines also have a vested interest in being assured of the origin (and, thereby, the quality) of their purchase, as well as holding proof of legitimate ownership of the cell line. In short, this emerging industry is in need of novel protocols for formally verifying the sale transaction of proprietary cell lines.

Moreover, cross-contamination or misidentification of cell lines due to poor handling, mislabeling, or procurement from dubious or undocumented sources is a rampant problem, resulting in innumerable financial and time losses. For example, a major German cell repository has reported that 20% of its human cell line stocks were cross contaminated with other cell lines, and the China Center for Type Culture Collection demonstrated that 85% of cell lines in their repository, supposedly established from primary isolates, were actually HeLa cells. Such issues undermine quality, repeatability and, ultimately, overall efficiency of medical research. Therefore, quality control and source verification provisions are paramount toward safeguarding against working with unsuitable cell line models and producing false data.

The cells, compounds, compositions, systems, and methods disclosed herein address these and other needs.

### SUMMARY

Disclosed herein is CRISPR Engineered Authentication of Mammalian Cells (CREAM-PUFs), a methodology which enables provenance attestation of cell lines through the use of the first genetic Physical Unclonable Functions (PUFs).

In one aspect, disclosed herein is a genetically modified cell comprising:
a nucleic acid comprising
a genetic barcode; and
an insertion or deletion mutation (indel mutation);
wherein the genetic barcode is adjacent to the indel mutation.

In some embodiments, the genetic barcode comprises a five nucleotide barcode. In some embodiments, the genetic barcode is selected from a genetic barcode library having at least 100 distinct genetic barcodes. In some embodiments, the genetic barcode is integrated into a genome of the cell via homologous recombination. In some embodiments, the genetic barcode is integrated into the genome of the cell via CRISPR/SpCas9-mediated homologous recombination.

In some embodiments, the nucleic acid further comprises a promoter. In some embodiments, the nucleic acid further comprises a truncated human cytomegalovirus (CMV) promoter. In some embodiments, the genetic barcode is located immediately upstream of the promoter.

In some embodiments, the nucleic acid further comprises a reporter gene. In some embodiments, the indel mutation is located within the reporter gene. In some embodiments, the indel mutation is located within an open reading frame of the reporter gene. In some embodiments, the reporter gene is a fluorescent reporter gene. In some embodiments, the fluorescent reporter gene is mKate.

In some embodiments, the indel mutation is stochastically generated. In some embodiments, the indel mutation is generated by a non-homologous end joining repair mechanism. In some embodiments, the indel mutation is from 1 to 16 nucleotides in length.

In some embodiments, the nucleic acid further comprises a selection marker gene. In some embodiments, the selection marker gene is an antibiotic resistance gene. In some embodiments, the antibiotic resistance gene is a hygromycin resistance gene.

In some embodiments, the cell is a mammalian cell. In some embodiments, the cell is a human cell. In some embodiments, the cell is from a HEK293 cell line, an HCT116 cell line, or a HeLa cell line. In some embodiments, the genetic barcode is integrated into an AAVS1 locus of the HEK293 cell line.

In some embodiments, the cell, prior to genetic modification, does not comprise the genetic barcode and/or the indel mutation.

In another aspect, disclosed herein is a genetically modified nucleic acid, comprising:
a genetic barcode;
a promoter, wherein the promoter is operably linked to a reporter gene; and
an insertion or deletion mutation (indel mutation), wherein the indel mutation is located within the reporter gene.

In some aspects, disclosed herein is a DNA vector comprising a nucleic acid as described herein. In some aspects, disclosed herein is a cell comprising a nucleic acid as described herein.

In some embodiments, the nucleic acid is integrated into a genome of the cell. In some embodiments, the cell, prior to integration of the nucleic acid into the genome of the cell, does not comprise the genetic barcode and/or the indel mutation.

In some aspects, disclosed herein is a method of manufacturing a cell line, comprising the steps of:
integrating a genetic barcode into a genome of a cell; and
integrating an insertion or deletion mutation (indel mutation) into the genome of the cell adjacent to the genetic barcode.

In some embodiments, the indel mutation is generated by non-homologous end joining (NHEJ) repair. In some embodiments, the indel mutation is generated via CRISPR/SpCas9-mediated non-homologous end joining (NHEJ) repair.

In some aspects, disclosed herein is a method for authenticating a cell line, comprising the steps of:
generating a database defining a set of linked genetic barcodes and insertion or deletion mutations (indel mutations) from a reference cell line;
extracting sequence information from a target cell line defining a set of linked genetic barcodes and indel mutations from the target cell line;
comparing the set of linked genetic barcodes and indel mutations from the target cell line to the database defining the set of linked genetic barcodes and indel mutations from the reference cell line; and
determining a matching probability between the target cell line and the reference cell line in the database.

In some embodiments, the matching probability is determined using a Bray-Curtis dissimilarity analysis.

### BRIEF DESCRIPTION OF THE DRAWINGS

The accompanying figures, which are incorporated in and constitute a part of this specification, illustrate several aspects described below.

FIG. 1. Provenance attestation protocols and pilot CREAM-PUF (CRISPR Engineered Authentication of Mammalian Cells-Physical Unclonable Functions). (A) The producer of a valuable cell line inserts a unique, robust and unclonable signature in each legitimately produced copy of this cell line. Upon thawing of a frozen sample and prior to its initial use, a customer who purchased a copy of the cell line can obtain this signature and communicate it to the producer who compares it against the signature database of legitimately produced copies of this cell line and, thereby, attests its provenance.

FIGS. 2A-2B. Overview of the CREAM-PUF generation process. (A) Schematic illustration of design of CREAM-PUFs. Barcodes were stably integrated into cell lines of interest, which were subsequently subjected to CRISPR/SpCas9 treatment to induce non-homologous end joining (NHEJ). The resulting two-dimensional mapping between barcodes and indels is evaluated for robustness and uniqueness. (B) Venn diagram comparing PUFs to other methods/technologies.

FIG. 3. Schematic illustration of implementation of CREAM-PUFs. Using CRISPR-Cas9 system, a set of synthetic constructs containing an array of 5-bp barcodes (5′-NNNNN-3′), constitutive fluorescent reporter and hygromycin resistance gene were stably integrated into the human AAVS1 safe harbor locus. Next, the cells were transiently transfected with CRISPR to induce NHEJ.

FIG. 4. Distribution of indels and barcodes that makeup a CREAM-PUF (Right) Frequency of detected barcodes. In total, 805 unique barcodes were observed. (Left) Frequency of detected indels. In total, 569 unique indels were observed. (Bottom) Barcode/Indel matrix. Heatmap presentation of the pilot CREAM-PUF matrix consisting of the 10 most frequently occurring barcodes and indels.

Sequences disclosed in FIG. 4:

(SEQ ID NO: 24)
AGGCAAGCCCTACGAGG;
(SEQ ID NO: 25)
TTCAAGTGCACATCCGAAGGCAAGCCCTACGAGG;
(SEQ ID NO: 26)
TTCAAGTGCACATCCGAGGGGAGG;
(SEQ ID NO: 27)
TTCAAGTGCACATCCGAGCGAAGGCAAGCCCTACGAGG;
(SEQ ID NO: 28)
TTCAAGTGCACATCCGAGG;
(SEQ ID NO: 29)
TTCAAGTGCACATCCGAGGGCAAGCCCTACGAGG;
(SEQ ID NO: 30)
TTCAAGTGCACATCCGAGGGGAAGGCAAGCCCTACGAGG;
(SEQ ID NO: 31)
TTCAAGTGCACATCCGAGGCAAGCCCTACGAGG;
(SEQ ID NO: 32)
TTCAAGTGCACATCGAAGGCAAGCCCTACGAGG;
(SEQ ID NO: 33)
TTCAAGTGCACATCCGAGGGAAGGCAAGCCCTACGAGG;
(SEQ ID NO: 34)
TTCAAGTGCACATCCGCGAAGGCAAGCCCTACGAGG.

FIG. 5. List of all CREAM-PUFs generated for this study. In this study, 2 independently barcoded HEK293 cell lines were each transfected with identical sgRNA in 3 separate instances, resulting in a total of 6 unique PUFs. In addition, a HCT116 cell line and a HeLa cell line, each with a distinct set of barcodes, were each transfected 6 times to generated 6 unique PUFs for each cell line. A portion of each PUF was subjected to one cycle of freeze-thaw (denoted as ft) before proceeding with next generation sequencing (NGS). In order to account for sequencing errors, PUFs from each barcoded cell line were also sequenced twice (denoted as r). Therefore, in total, 50 samples were sequenced via NGS, each resulting in a unique matrix of barcode/indel frequencies. To assess robustness to NGS measurement error, the matrix for each PUF i.j is compared against the matrix of its technical replicate PUF i.jr (i={1,2,3,4}). Similarly, to assess robustness to the freeze-thaw process, the matrix for each PUF i.j is compared against the matrix of its freeze-thaw counterpart PUF i.jft (i={1,2,3,4}). To assess uniqueness, the matrices of all PUF i.j (i={1,2,3,4}) are compared pairwise.

FIGS. 6A-6D. Qualitative assessment of CREAM-PUFs generated using HEK293. (A˜D) Frequencies of barcode-indel addresses consisting of the 5 most commonly observed barcodes and indels (Left) and heatmap based on the same data but expanded to the top 30 most commonly observed barcodes and indels (Right) for a given PUF and its freeze-thaw counterparts and technical replicates (if applicable). The green dashed square on the heatmap represents the data shown on the table. Data shown in (A, B) are barcode-indel addresses for PUF1.1 (A) and PUF2.1 (B) with their respective freeze-thaw counterpart and technical replicate. Data shown in (C, D) are barcode-indel addresses for PUF1.2, PUF1.3 (C), PUF2.2, PUF2.3 (D) with their respective freeze-thaw counterpart.

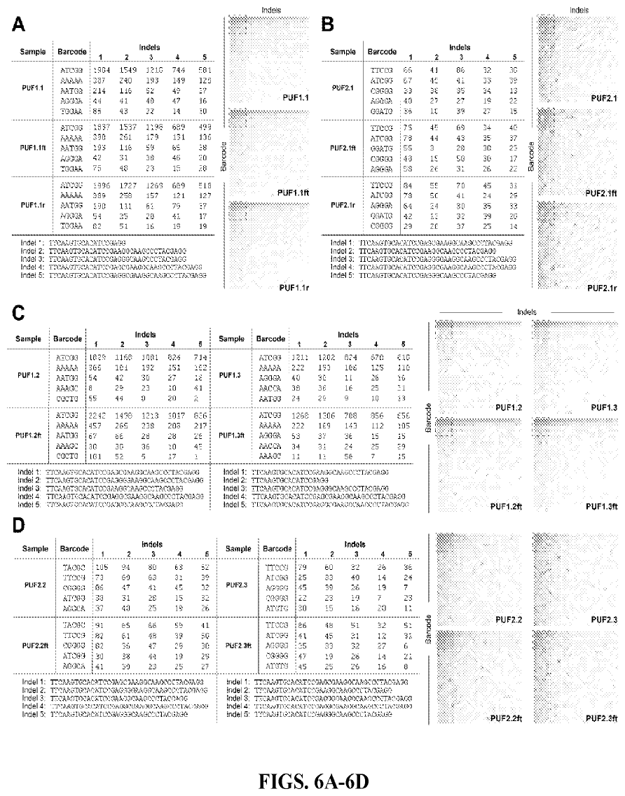

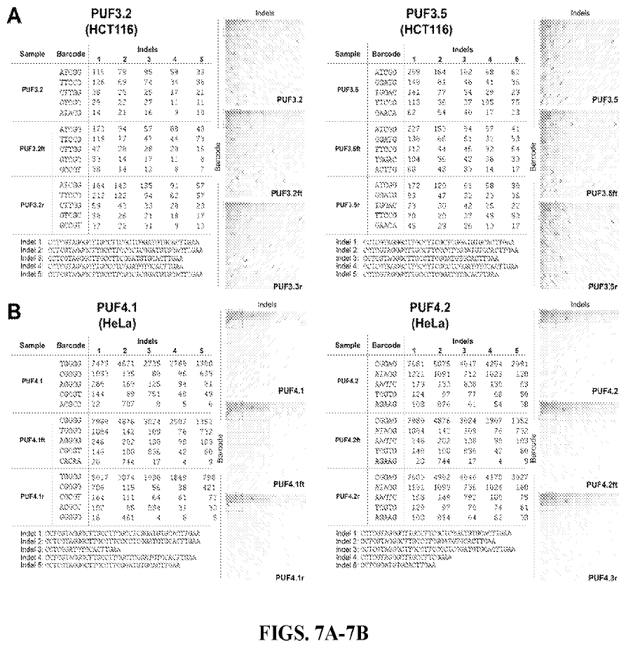

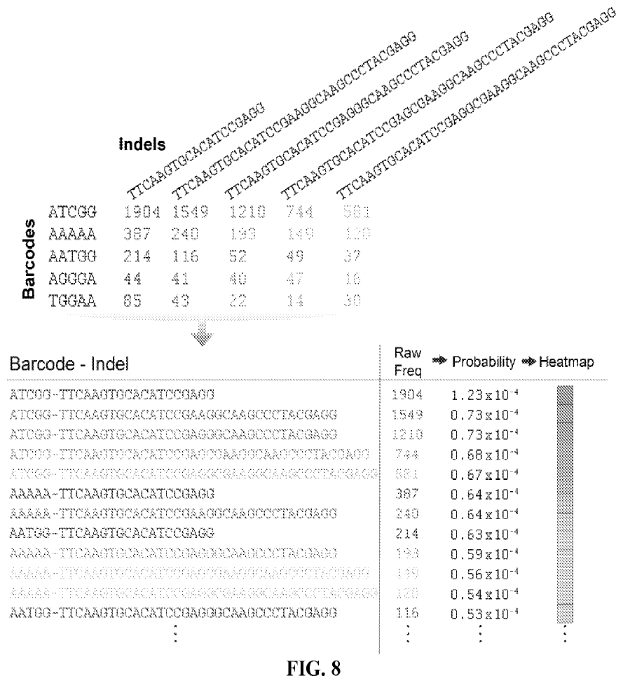

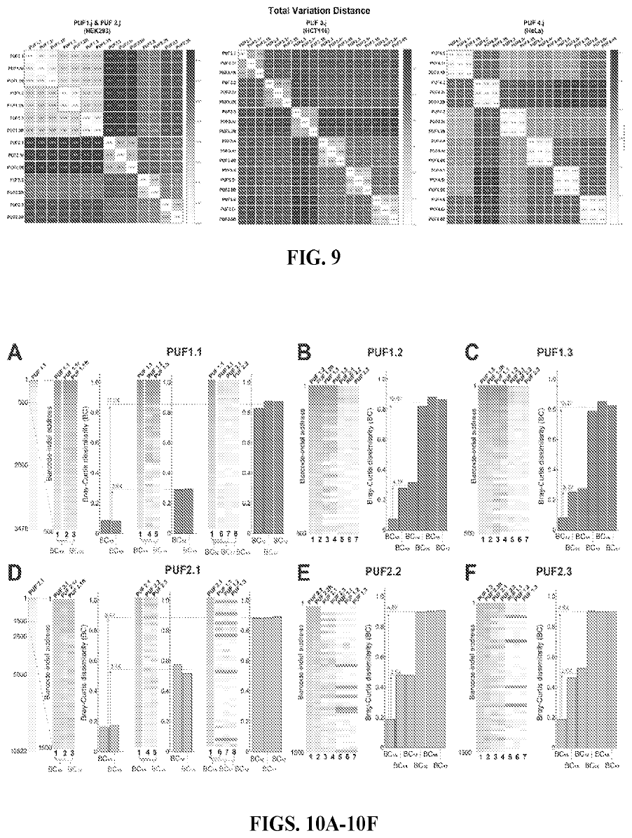

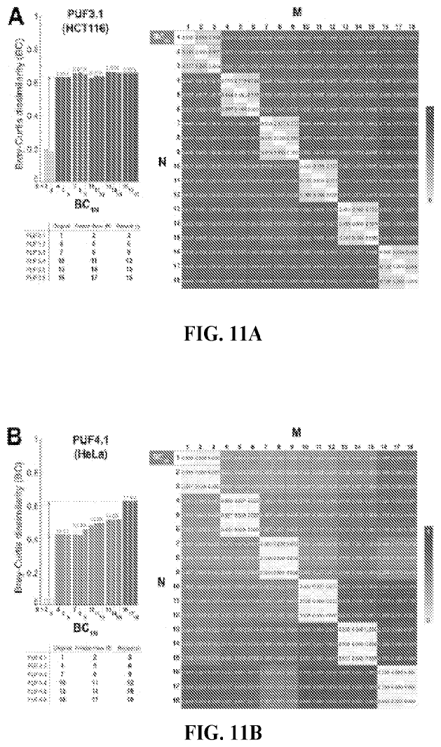

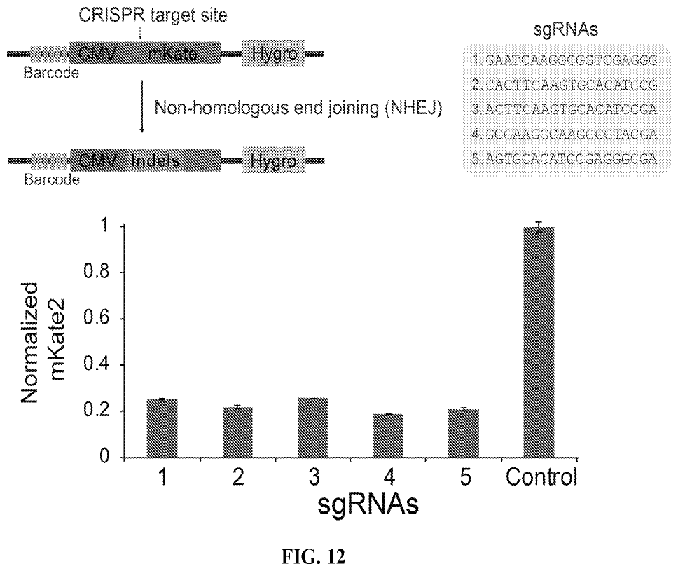

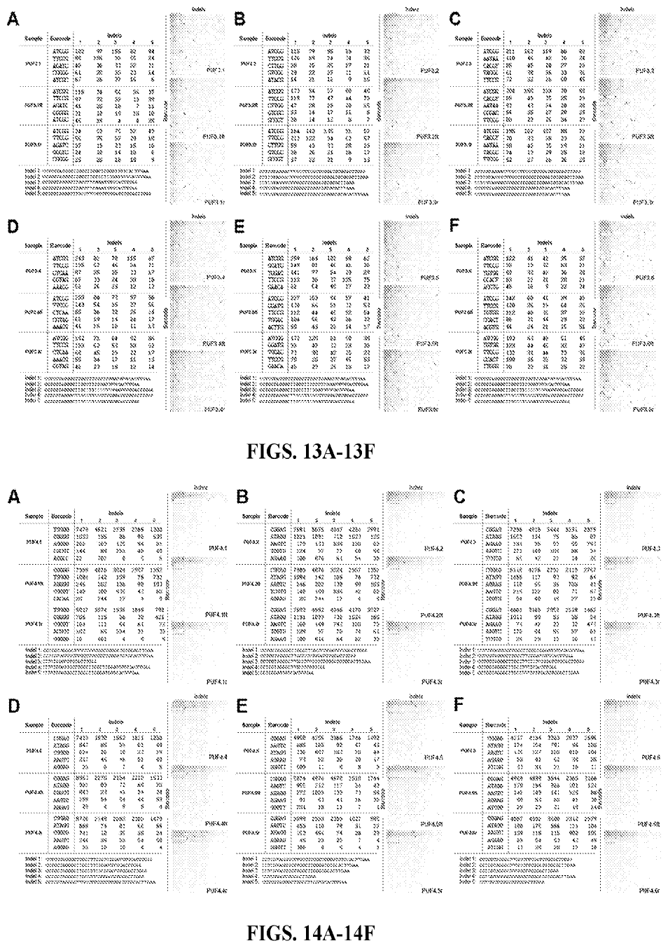

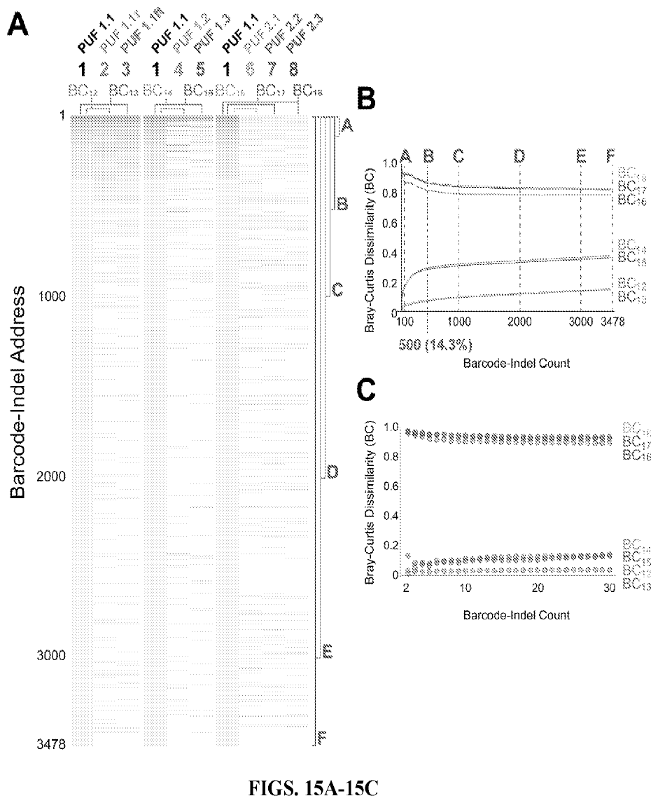

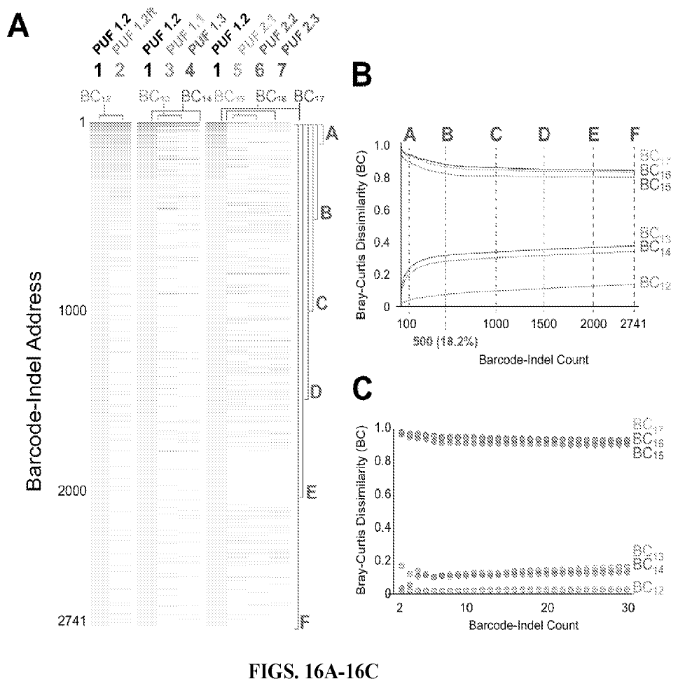

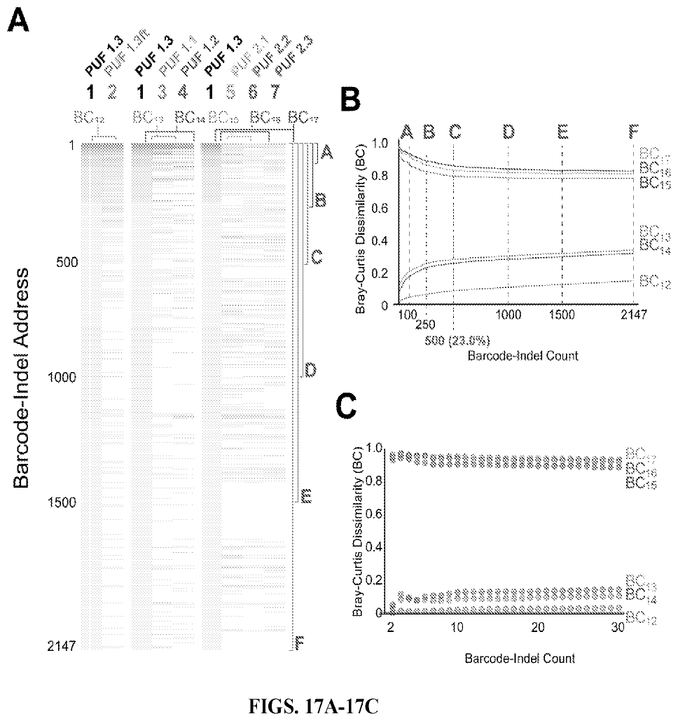

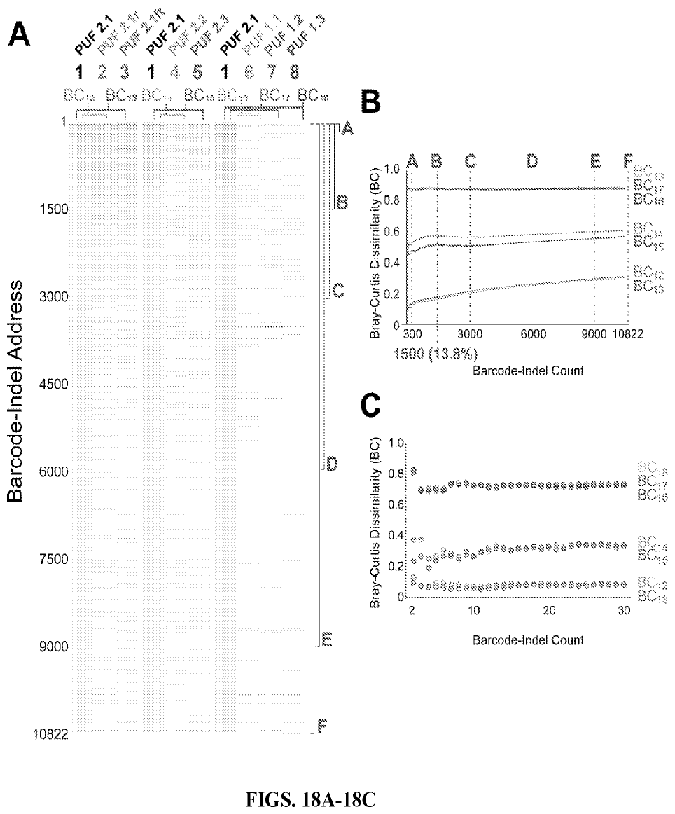

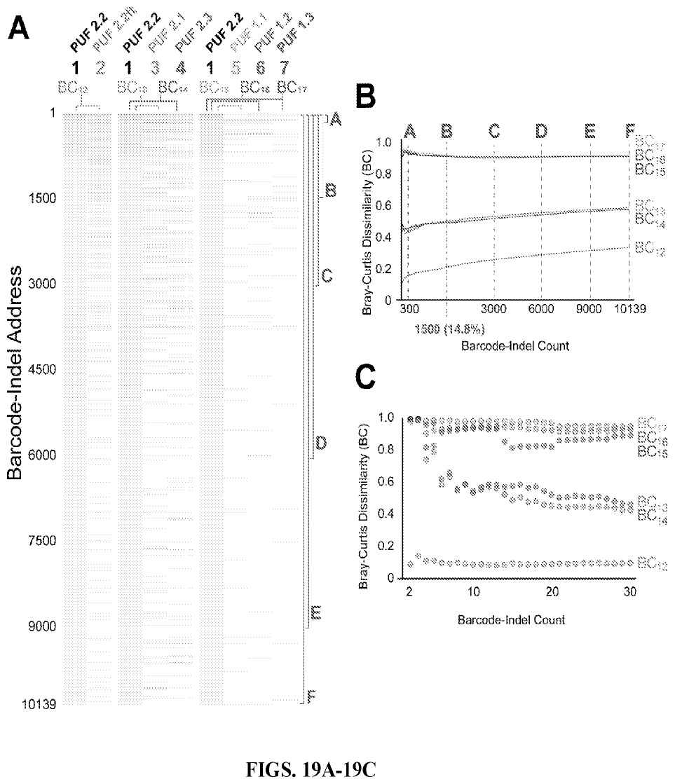

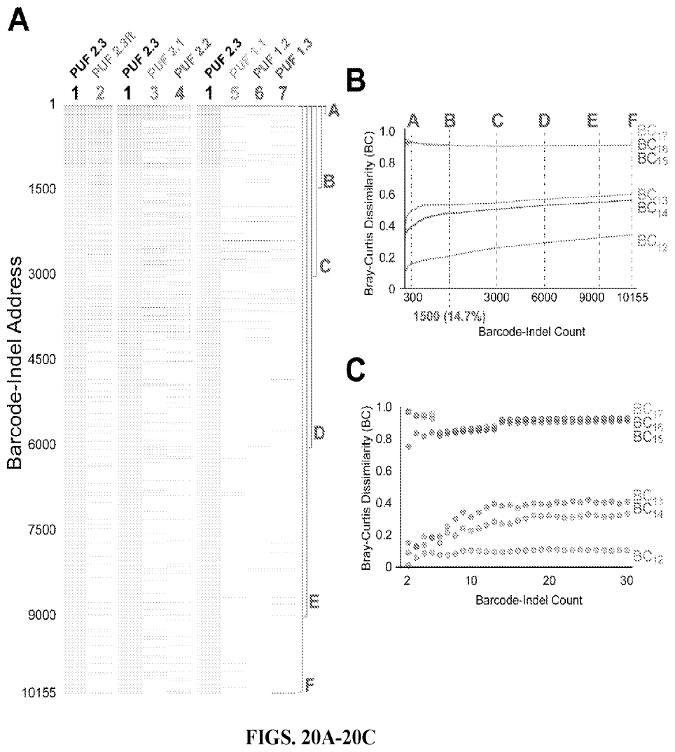

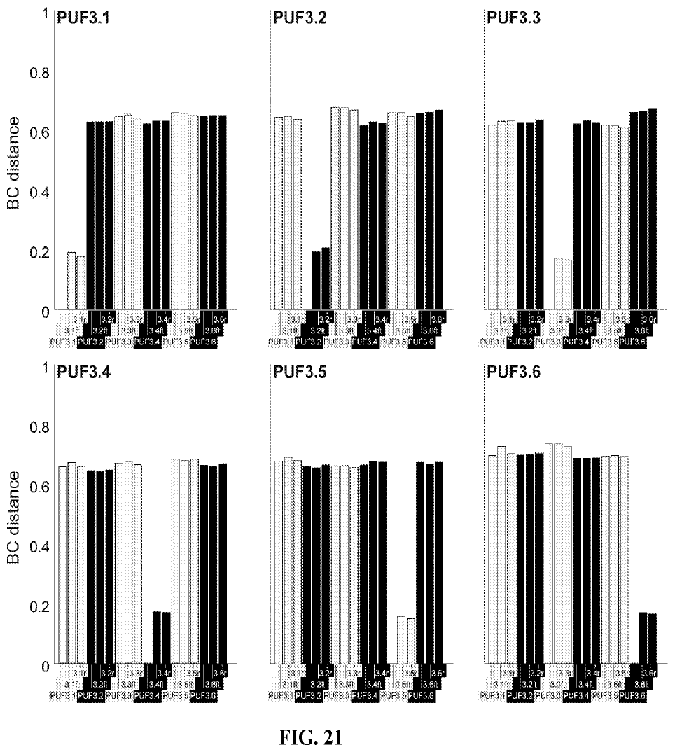

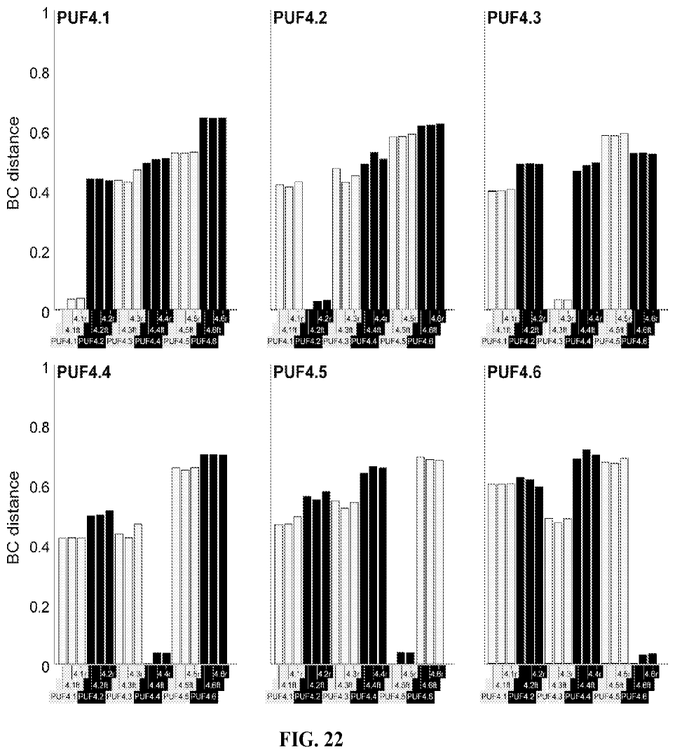

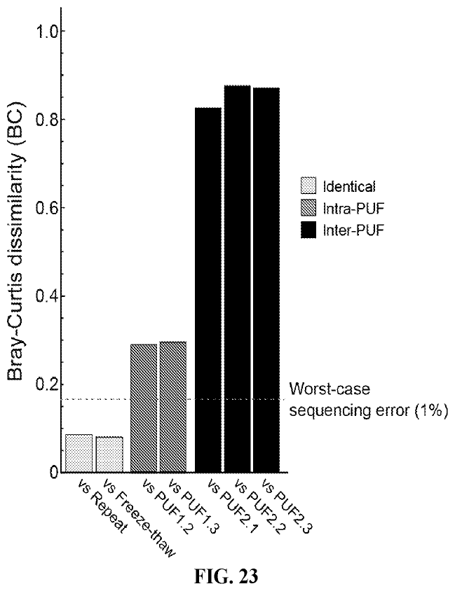

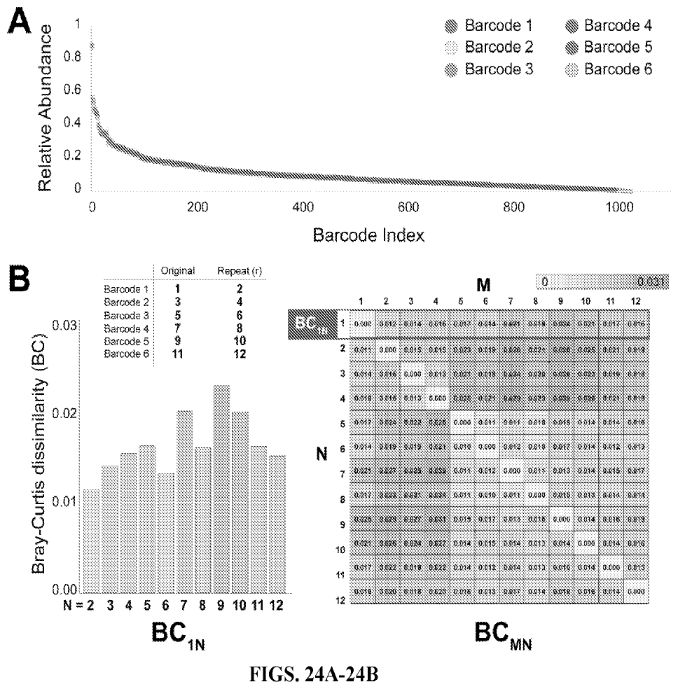

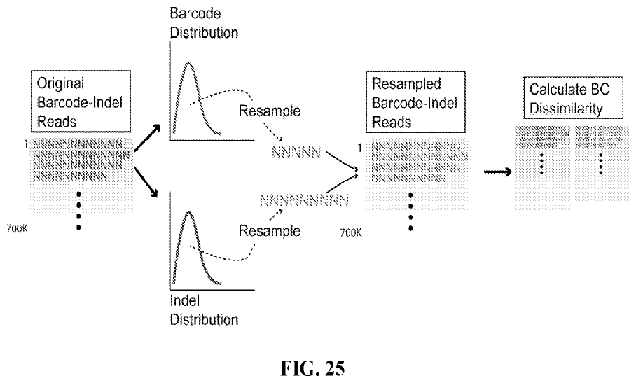

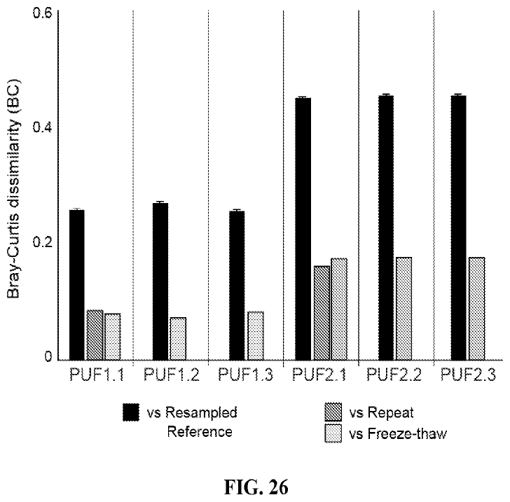

Sequences disclosed in FIG. 6A:

Indel 1:
(SEQ ID NO: 35)
TTCAAGTGCACATCCGAGG;
Indel 2:
(SEQ ID NO: 36)
TTCAAGTGCACATCCGAAGGCAAGCCCTACGAGG;
Indel 3:
(SEQ ID NO: 37)
TTCAAGTGCACATCCGAGGGCAAGCCCTACGAGG;
Indel 4:
(SEQ ID NO: 38)
TTCAAGTGCACATCCGAGCGAAGGCAAGCCCTACGAGG;
Indel 5:
(SEQ ID NO: 39)
TTCAAGTGCACATCCGAGGCGAAGGCAAGCCCTACGAGG.

Sequences disclosed in FIG. 6B:

Indel 1:
(SEQ ID NO: 40)
TTCAAGTGCACATCCGAGCGAAGGCAAGCCCTACGAGG;
Indel 2:
(SEQ ID NO: 41)
TTCAAGTGCACATCCGAAGGCAAGCCCTACGAGG;
Indel 3:
(SEQ ID NO: 42)
TTCAAGTGCACATCCGAGGGGAAGGCAAGCCCTACGAGG;
Indel 4:
(SEQ ID NO: 43)
TTCAAGTGCACATCCGAGGCGAAGGCAAGCCCTACGAGG;
Indel 5:
(SEQ ID NO: 44)
TTCAAGTGCACATCCGAGGGCAAGCCCTACGAGG.

Sequences disclosed in FIG. 6C:

Section 1
Indel 1:
(SEQ ID NO: 45)
TTCAAGTGCACATCCGAGCGAAGGCAAGCCCTACGAGG;
Indel 2:
(SEQ ID NO: 46)
TTCAAGTGCACATCCGAGGGGAAGGCAAGCCCTACGAGG;
Indel 3:
(SEQ ID NO: 47)
TTCAAGTGCACATCCGAAGGCAAGCCCTACGAGG;
Indel 4:
(SEQ ID NO: 48)
TTCAAGTGCACATCCGAGGCGAAGGCAAGCCCTACGAGG;
Indel 5:
(SEQ ID NO: 49)
TTCAAGTGCACATCCGAGGGCAAGCCCTACGAGG.
Section 2
Indel 1:
(SEQ ID NO: 50)
TTCAAGTGCACATCCGAAGGCAAGCCCTACGAGG;
Indel 2:
(SEQ ID NO: 51)
TTCAAGTGCACATCCGAGG;
Indel 3:
(SEQ ID NO: 52)
TTCAAGTGCACATCCGAGGGCAAGCCCTACGAGG;
Indel 4:
(SEQ ID NO: 53)
TTCAAGTGCACATCCGAGCGAAGGCAAGCCCTACGAGG;
Indel 5:
(SEQ ID NO: 54)
TTCAAGTGCACATCCGAGGCGAAGGCAAGCCCTACGAGG.

Sequences disclosed in FIG. 6D:

Section 1
Indel 1:
(SEQ ID NO: 55)
TTCAAGTGCACATCCGAGCGAAGGCAAGCCCTACGAGG;
Indel 2:
(SEQ ID NO: 56)
TTCAAGTGCACATCCGAGGGGAAGGCAAGCCCTACGAGG;
Indel 3:
(SEQ ID NO: 57)
TTCAAGTGCACATCCGAAGGCAAGCCCTACGAGG;
Indel 4:
(SEQ ID NO: 58)
TTCAAGTGCACATCCGAGGCGAAGGCAAGCCCTACGAGG;
Indel 5:
(SEQ ID NO: 59)
TTCAAGTGCACATCCGAGGGCAAGCCCTACGAGG.
Section 2
Indel 1:
(SEQ ID NO: 60)
TTCAAGTGCACATCCGAGCGAAGGCAAGCCCTACGAGG;
Indel 2:
(SEQ ID NO: 61)
TTCAAGTGCACATCCGAAGGCAAGCCCTACGAGG;
Indel 3:
(SEQ ID NO: 62)
TTCAAGTGCACATCCGAGGGGAAGGCAAGCCCTACGAGG;
Indel 4:
(SEQ ID NO: 63)
TTCAAGTGCACATCCGAGGCGAAGGCAAGCCCTACGAGG;
Indel 5:
(SEQ ID NO: 64)
TTCAAGTGCACATCCGAGGGCAAGCCCTACGAGG.

FIGS. 7A-7B. Qualitative assessment of CREAM-PUFs generated using HCT116 and HeLa. Qualitative analysis of PUFs as shown in FIG. 6, with HCT116 (A) and HeLa (B). In both cell types, heatmaps of barcode-indel addresses from intra-PUFs were visually similar, via different between inter-PUFs.

Sequences disclosed in FIG. 7A:

Section 1
Indel 1:
(SEQ ID NO: 65)
CCTCGTAGGGCTTGCCTTCGCTCGGATGTGCACTTGAA;
Indel 2:
(SEQ ID NO: 66)
CCTCGTAGGGCTTGCCTTCCCCTCGGATGTGCACTTGAA;
Indel 3:
(SEQ ID NO: 67)
CCTCGTAGGGCTTGCCTTCGGATGTGCACTTGAA;
Indel 4:
(SEQ ID NO: 68)
CCTCGTAGGGCTTGCCCTCGGATGTGCACTTGAA;
Indel 5:
(SEQ ID NO: 69)
CCTCGTAGGGCTTGCCTTCGCCTCGGATGTGCACTTGAA.
Section 2
Indel 1:
(SEQ ID NO: 70)
CCTCGTAGGGCTTGCCTTCGCTCGGATGTGCACTTGAA;
Indel 2:
(SEQ ID NO: 71)
CCTCGTAGGGCTTGCCTTCCCCTCGGATGTGCACTTGAA;
Indel 3:
(SEQ ID NO: 72)
CCTCGTAGGGCTTGCCTTCGGATGTGCACTTGAA;
Indel 4:
(SEQ ID NO: 73)
CCTCGTAGGGCTTGCCTTCGCCTCGGATGTGCACTTGAA;
Indel 5:
(SEQ ID NO: 74)
CCTCGTAGGGCTTGCCCTCGGATGTGCACTTGAA.

Sequences disclosed in FIG. 7B:

Section 1
Indel 1:
(SEQ ID NO: 75)
CCTCGTAGGGCTTGCCTTCGCCTCGGATGTGCACTTGAA;
Indel 2:
(SEQ ID NO: 76)
CCTCGTAGGGCTTGCCTTCCCCTCGGATGTGCACTTGAA;
Indel 3:
(SEQ ID NO: 77)
CCTCGGATGTGCACTTGAA;
Indel 4:
(SEQ ID NO: 78)
CCTCGTAGGGCTTGCCTTCGCTCGGATGTGCACTTGAA;
Indel 5:
(SEQ ID NO: 79)
CCTCGTAGGGCTTGCCTTCGGATGTGCACTTGAA.
Section 2
Indel 1:
(SEQ ID NO: 80)
CCTCGTAGGGCTTGCCTTCGCTCGGATGTGCACTTGAA;
Indel 2:
(SEQ ID NO: 81)
CCTCGTAGGGCTTGCCCTCGGATGTGCACTTGAA;
Indel 3:
(SEQ ID NO: 82)
CCTCGTAGGGCTTGCCTTCCCCTCGGATGTGCACTTGAA;
Indel 4:
(SEQ ID NO: 83)
CCTCGTAGGGCTTGCCTTCGGAA;
Indel 5:
(SEQ ID NO: 84)
CCTCGGATGTGCACTTGAA.

FIG. 8. Quantitative assessment of CREAM-PUFs. To quantitatively assess the effectiveness of CREAM-PUFs, the NGS result was converted to a frequency-based array of barcode-indel combinations. The corresponding probability density functions were then calculated to enable comparison between samples.

Sequences disclosed in FIG. 8:

(SEQ ID NO: 85)
TTCAAGTGCACATCCGAGG;
(SEQ ID NO: 86)
TTCAAGTGCACATCCGAAGGCAAGCCCTACGAGG;
(SEQ ID NO: 87)
TTCAAGTGCACATCCGAGGGCAAGCCCTACGAGG;
(SEQ ID NO: 88)
TTCAAGTGCACATCCGAGCGAAGGCAAGCCCTACGAGG;
(SEQ ID NO: 89)
TTCAAGTGCACATCCGAGGCGAAGGCAAGCCCTACGAGG;
(SEQ ID NO: 90)
ATCGGTTCAAGTGCACATCCGAGG;
(SEQ ID NO: 91)
ATCGGTTCAAGTGCACATCCGAAGGCAAGCCCTACGAGG;
(SEQ ID NO: 92)
ATCGGTTCAAGTGCACATCCGAGGGCAAGCCCTACGAGG;
(SEQ ID NO: 93)
ATCGGTTCAAGTGCACATCCGAGCGAAGGCAAGCCCTACGAGG;
(SEQ ID NO: 94)
ATCGGTTCAAGTGCACATCCGAGGCGAAGGCAAGCCCTACGAGG;
(SEQ ID NO: 95)
AAAAATTCAAGTGCACATCCGAGG;
(SEQ ID NO: 96)
AAAAATTCAAGTGCACATCCGAAGGCAAGCCCTACGAGG;
(SEQ ID NO: 97)
AATGGTTCAAGTGCACATCCGAGG;
(SEQ ID NO: 98)
AAAAATTCAAGTGCACATCCGAGGGCAAGCCCTACGAGG;
(SEQ ID NO: 99)
AAAAATTCAAGTGCACATCCGAGCGAAGGCAAGCCCTACGAGG;
(SEQ ID NO: 100)
AAAAATTCAAGTGCACATCCGAGGCGAAGGCAAGCCCTACGAGG;
(SEQ ID NO: 101)
AATGGTTCAAGTGCACATCCGAAGGCAAGCCCTACGAGG.

FIG. 9. Quantitative assessment of CREAM-PUFs using total variation distance. Pairwise Total Variation Distances between all PUFs were calculated in samples derived from HEK293, HCT116, and HeLa cells.

FIGS. 10A-10F. Quantitative assessment of HEK293-derived CREAM-PUFs using Bray-Curtis dissimilarity (A) The difference between each pair of barcode-indel arrays is quantified using the Bray-Curtis dissimilarity method. Prior to calculating the Bray-Curtis value, the barcode-indel arrays are trimmed down to approximately 15% of the total dataset (left). Then, the Bray-Curtis dissimilarity calculations against the reference PUF (i.e., PUF1.1) are made for 3 groups: 1) technical replicates (Left), 2) PUFs originating from the same barcoded cell line (Center) and 3) PUFs originating from a different barcoded cell line (Right). Bray-Curtis values shown in (B, C) are results of an identical analysis as in (A) but using PUF1.2 and PUF1.3 as the reference, respectively. Bray-Curtis values shown in (D, E and F) are analogous results to (A, B and C) respectively, using PUF2.1, PUF2.2 and PUF.2.3 as the reference, respectively. Again, approximately 15% of the total dataset is used.

FIGS. 11A-11B. Quantitative assessment of HCT116- and HeLa-derived CREAM-PUFs using Bray-Curtis dissimilarity (A, Left) Comparison of Bray-Curtis dissimilarities for a single PUF (PUF3.1) generated in HCT116 against 17 other PUFs generated in the same cell line. The barcode-indel arrays are trimmed down as described in previously in FIG. 10. (A, Right) Matrix of pair-wise Bray-Curtis dissimilarity for all 18 PUFs generated in HCT116. Results shown in (B) are same analysis as before, with PUFs generated in HeLa cell line (PUF4.1).

FIG. 12. Implementation of CREAM-PUFs in HEK293 cells. Five sgRNAs were designed to target the Open Reading Frames (ORFs) of the mKate2 construct, and demonstrated comparable efficiencies using in vitro fluorescence reporter assays.

Sequences disclosed in FIG. 12:

(SEQ ID NO: 102)
GAATCAAGGCGGTCGAGGG;
(SEQ ID NO: 103)
CACTTCAAGTGCACATCCG;
(SEQ ID NO: 104)
ACTTCAAGTGCACATCCGA;
(SEQ ID NO: 105)
GCGAAGGCAAGCCCTACGA;
(SEQ ID NO: 106)
AGTGCACATCCGAGGGCGA.

FIGS. 13A-13F. Implementation of CREAM-PUFs in HCT116 cells. Qualitative assessment of CREAM-PUFs generated using HCT116. (A˜E) Frequencies of barcode-indel addresses consisting of the 5 most commonly observed barcodes and indels (Left) and heatmap based on the same data but expanded to the top 30 most commonly observed barcodes and indels (Right) for a given PUF and its freeze-thaw counterparts and technical replicates. The green dashed square on the heatmap represents the data shown on the table. Data shown in (A) are barcode-indel addresses for PUF3.1 with their respective freeze-thaw counterpart and technical replicate. Data shown in (B˜E) are for PUFs 3.2 to 3.6, respectively, which are produced identically to PUF3.1 using the same barcoded cell line and same sgRNA to introduce indels.

Sequences disclosed in FIG. 13A:

Indel 1:
(SEQ ID NO: 107)
CCTCGTAGGGCTTGCCTTCGCTCGGATGTGCACTTGAA;
Indel 2:
(SEQ ID NO: 108)
CCTCGTAGGGCTTGCCTTCCCCTCGGATGTGCACTTGAA;
Indel 3:
(SEQ ID NO: 109)
CCTCGTAGGGCTTGCCTTCGGATGTGCACTTGAA;
Indel 4:
(SEQ ID NO: 110)
CCTCGTAGGGCTTGCCCTCGGATGTGCACTTGAA;
Indel 5:
(SEQ ID NO: 111)
CCTCGTAGGGCTTGCCTTCGCCTCGGATGTGCACTTGAA.

Sequences disclosed in FIG. 13B:

Indel 1:
(SEQ ID NO: 112)
CCTCGTAGGGCTTGCCTTCGCTCGGATGTGCACTTGAA;
Indel 2:
(SEQ ID NO: 113)
CCTCGTAGGGCTTGCCTTCCCCTCGGATGTGCACTTGAA;
Indel 3:
(SEQ ID NO: 114)
CCTCGTAGGGCTTGCCTTCGGATGTGCACTTGAA;
Indel 4:
(SEQ ID NO: 115)
CCTCGTAGGGCTTGCCCTCGGATGTGCACTTGAA;
Indel 5:
(SEQ ID NO: 116)
CCTCGTAGGGCTTGCCTTCGCCTCGGATGTGCACTTGAA.

Sequences disclosed in FIG. 13C:

Indel 1:
(SEQ ID NO: 117)
CCTCGTAGGGCTTGCCTTCGCTCGGATGTGCACTTGAA;
Indel 2:
(SEQ ID NO: 118)
CCTCGTAGGGCTTGCCTTCCCCTCGGATGTGCACTTGAA;
Indel 3:
(SEQ ID NO: 119)
CCTCGTAGGGCTTGCCTTCGCCTCGGATGTGCACTTGAA;
Indel 4:
(SEQ ID NO: 120)
CCTCGTAGGGCTTGCCTTCGGATGTGCACTTGAA;
Indel 5:
(SEQ ID NO: 121)
CCTCGTAGGGCTTGCCCTCGGATGTGCACTTGAA.

Sequences disclosed in FIG. 13D:

Indel 1:
(SEQ ID NO: 122)
CCTCGTAGGGCTTGCCTTCGCTCGGATGTGCACTTGAA;
Indel 2:
(SEQ ID NO: 123)
CCTCGTAGGGCTTGCCTTCGGATGTCACTTGAA;
Indel 3:
(SEQ ID NO: 124)
CCTCGTAGGGCTTGCCTTCCCCTCGGATGTGCACTTGAA;
Indel 4:
(SEQ ID NO: 125)
CCTCGTAGGGCTTGCCTTCGCCTCGGATGTGCACTTGAA;
Indel 5:
(SEQ ID NO: 126)
CCTCGTAGGGCTTGCCCTCGGATGTGCACTTGAA.

Sequences disclosed in FIG. 13E:

Indel 1:
(SEQ ID NO: 127)
CCTCGTAGGGCTTGCCTTCGCTCGGATGTGCACTTGAA;
Indel 2:
(SEQ ID NO: 128)
CCTCGTAGGGCTTGCCTTCCCCTCGGATGTGCACTTGAA;
Indel 3:
(SEQ ID NO: 129)
CCTCGTAGGGCTTGCCTTCGGATGTGCACTTGAA;
Indel 4:
(SEQ ID NO: 130)
CCTCGTAGGGCTTGCCTTCGCCTCGGATGTGCACTTGAA;
Indel 5:
(SEQ ID NO: 131)
CCTCGTAGGGCTTGCCCTCGGATGTGCACTTGAA.

Sequences disclosed in FIG. 13F:

Indel 1:
(SEQ ID NO: 132)
CCTCGTAGGGCTTGCCTTCGCTCGGATGTGCACTTGAA;
Indel 2:
(SEQ ID NO: 133)
CCTCGTAGGGCTTGCCTTCGGATGTGCACTTGAA;
Indel 3:
(SEQ ID NO: 134)
CCTCGTAGGGCTTGCCTTCCCCTCGGATGTGCACTTGAA;
Indel 4:
(SEQ ID NO: 135)
CCTCGTAGGGCTTGCCTTCGCCTCGGATGTGCACTTGAA;
Indel 5:
(SEQ ID NO: 136)
CCTCGTAGGGCTTGCCCTCGGATGTGCACTTGAA.

FIGS. 14A-14F. Implementation of CREAM-PUFs in HeLa cells. Qualitative assessment of CREAM-PUFs generated using HeLa. See FIG. 13 for detailed description.

Sequences disclosed in FIG. 14A:

Indel 1:
(SEQ ID NO: 137)
CCTCGTAGGGCTTGCCTTCGCCTCGGATGTGCACTTGAA;
Indel 2:
(SEQ ID NO: 138)
CCTCGTAGGGCTTGCCTTCCCCTCGGATGTGCACTTGAA;
Indel 3:
(SEQ ID NO: 139)
CCTCGGATGTGCACTTGAA;
Indel 4:
(SEQ ID NO: 140)
CCTCGTAGGGCTTGCCTTCGCTCGGATGTGCACTTGAA;
Indel 5:
(SEQ ID NO: 141)
CCTCGTAGGGCTTGCCTTCGGATGTGCACTTGAA.

Sequences disclosed in FIG. 14B:

Indel 1:
(SEQ ID NO: 142)
CCTCGTAGGGCTTGCCTTCGCTCGGATGTGCACTTGAA;
Indel 2:
(SEQ ID NO: 143)
CCTCGTAGGGCTTGCCCTCGGATGTGCACTTGAA;
Indel 3:
(SEQ ID NO: 144)
CCTCGTAGGGCTTGCCTTCCCCTCGGATGTGCACTTGAA;
Indel 4:
(SEQ ID NO: 145)
CCTCGTAGGGCTTGCCTTCGGAA;
Indel 5:
(SEQ ID NO: 146)
CCTCGGATGTGCACTTGAA.

Sequences disclosed in FIG. 14C:

Indel 1:
(SEQ ID NO: 147)
CCTCGTAGGGCTTGCCTTCGCTCGGATGTGCACTTGAA;
Indel 2:
(SEQ ID NO: 148)
CCTCGTAGGGCTTGCCTTCGGATGTGCACTTGAA;
Indel 3:
(SEQ ID NO: 149)
CCTCGTAGGGCTTGCCTTCCCTCGGATGTGCACTTGAA;
Indel 4:
(SEQ ID NO: 150)
CCTCGTAGGGCTTGCCTTCGCACTTGAA;
Indel 5:
(SEQ ID NO: 151)
CCTCGTAGGGCTTGCCTTCGCGGATGTGCACTTGAA.

Sequences disclosed in FIG. 14D:

Indel 1:
(SEQ ID NO: 152)
CCTCGTAGGGCTTGCCTTCGCTCGGATGTGCACTTGAA;
Indel 2:
(SEQ ID NO: 153)
CCTCGTAGGGCTTGCCTTCGGATGTGCACTTGAA;
Indel 3:
(SEQ ID NO: 154)
CCTCGTAGGGCTTGCCTTCGCGGATGTGCACTTGAA;
Indel 4:
(SEQ ID NO: 155)
CCTCGTAGGGCTTGCCTTCGGGATGTGCACTTGAA;
Indel 5:
(SEQ ID NO: 156)
CCTCGTAGGGCTTGCCTTCCCCTCGGATGTGCACTTGAA.

Sequences disclosed in FIG. 14E:

Indel 1:
(SEQ ID NO: 157)
CCTCGTAGGGCTTGCCTTCGCTCGGATGTGCACTTGAA;
Indel 2:
(SEQ ID NO: 158)
CCTCGTAGGGCTTGCCTTCGGATGTGCACTTGAA;
Indel 3:
(SEQ ID NO: 159)
CCTCGTAGGGCTTGCCTTCGGTGCACTTGAA;
Indel 4:
(SEQ ID NO: 160)
CCTCGGATGTGCACTTGAA;
Indel 5:
(SEQ ID NO: 161)
CCTCGTAGGGCTTGCCTTCGTGCACTTGAA.

Sequences disclosed in FIG. 14F:

Indel 1:
(SEQ ID NO: 162)
CCTCGTAGGGCTTGCCTTCGATCTGCACTTGAA;
Indel 2:
(SEQ ID NO: 163)
CCTCGTAGGGCTTGCCTTCGGATCTGCACTTGAA;
Indel 3:
(SEQ ID NO: 164)
CCTCGTAGGGCTTGCCTTCGCACTTGAA;
Indel 4:
(SEQ ID NO: 165)
CCTCGTAGGGCTTGCCCTCGGATGTGCACTTGAA;
Indel 5:
(SEQ ID NO: 166)
CCTCGCTCGGATGTGCACTTGAA.

FIGS. 15A-15C. Calculation of Bray-Curtis dissimilarities using PUF 1.1 as reference with varying sampling rate. (A) To calculate the Bray-Curtis value between 2 PUFs, the NGS results are first turned into an array of barcode-indel combinations. After sorting the array of the reference PUF based on frequency of occurrence, entries of the other arrays are then sorted to match this order. (B) The Bray-Curtis value between the reference and another PUF based on the size of the barcode-indel list used in the calculation, from 2 to the size of the reference sample. Purple letters indicate section of the array shown in (A) that corresponds to the visual representation of the list used in the calculation. The barcode-indel count shown in red indicates the list size used for analysis in the main text. (C) The Bray-Curtis dissimilarity based on the size of the barcode-indel list used to obtain the distance, from 2 to 30.

FIGS. 16A-16C. Calculation of Bray-Curtis dissimilarities using PUF 1.2 as reference with varying sampling rate. Refer to FIGS. 15A-15C for a detailed description.

FIGS. 17A-17C. Calculation of Bray-Curtis dissimilarities using PUF 1.3 as reference with varying sampling rate. Refer to FIGS. 15A-15C for a detailed description.

FIGS. 18A-18C. Calculation of Bray-Curtis dissimilarities using PUF 2.1 as reference with varying sampling rate. Refer to FIGS. 15A-15C for a detailed description.

FIGS. 19A-19C. Calculation of Bray-Curtis dissimilarities using PUF 2.2 as reference with varying sampling rate. Refer to FIGS. 15A-15C for a detailed description.

FIGS. 20A-20C. Calculation of Bray-Curtis dissimilarities using PUF 2.3 as reference with varying sampling rate. Refer to FIGS. 15A-15C for a detailed description.

FIG. 21. Quantitative assessment of HCT116-derived CREAM-PUFs using Bray-Curtis dissimilarity. Comparison of Bray-Curtis dissimilarities for a single PUF3.i (i={1,2,3,4,5,6}) generated in HCT116 against 17 other PUFs generated in the same cell line.

FIG. 22. Quantitative assessment of HeLa-derived CREAM-PUFs using Bray-Curtis dissimilarity. Comparison of Bray-Curtis dissimilarities for a single PUF4.i (i={1,2,3,4,5,6}) generated in HeLa against 17 other PUFs generated in the same cell line.

FIG. 23. Simulated maximum Bray-Curtis dissimilarity from sequencing error for PUFs. To obtain the worst-case Bray-Curtis values from sequencing error, each PUF barcode-indel sequencing data were mutated in silico using an error rate of 1% per base. The resulting dataset was then used to calculate the Bray-Curtis value against the original sequence and the technical replicates of the original sequence (repeat and freeze-thaw). The value shown for worst-case sequencing error is an average of 100 different simulations.

FIGS. 24A-24B. Barcode library alone does not satisfy the uniqueness requirement of PUFs. A 5-nucleotide barcode library was stably integrated into the AAVS1 locus of HEK293 cells in 6 parallel trials. (A) The relative abundances of stably integrated barcodes in 6 replicates. (B) The Bray-Curtis dissimilarity values between barcode 1 and all other 6 samples and their NGS sequencing replicates (left) and of any given pair of all barcodes (right). Note the intra-sample dissimilarities generally overlapped with those of inter-samples, thus violating the uniqueness requirement of PUFs.

FIG. 25. Procedure for generating resampled Barcode-Indel reads and corresponding BC dissimilarity

FIG. 26. Bray-Curtis dissimilarities for intra-PUFs and simulated inter-PUFs.

### DETAILED DESCRIPTION

Disclosed herein is a novel methodology, namely CRISPR-Engineered Attestation of Mammalian Cells using Physical Unclonable Functions (CREAM-PUFs), which can serve as the cornerstone for formally verifying transactions in cell line distribution networks. A PUF is a physical entity which provides a measurable output that can be used as a unique and irreproducible identifier for the artifact wherein it is embedded. Popularized by the electronics industry, silicon PUFs leverage the inherent physical variations of semiconductor manufacturing to establish intrinsic security primitives for attesting integrated circuits. Owing to the stochastic nature of these variations and the multitude of steps involved, photo-lithographically manufactured silicon PUFs are impossible to reproduce (thus unclonable). Inspired by the success of silicon PUFs, it was sought to exploit a combination of sequence-restricted barcodes and the inherent stochasticity of CRISPR-induced non-homologous end joining DNA error repair to create the first generation of genetic physical unclonable functions in three distinct human cells (HEK293, HCT116, and HeLa). It was demonstrated that these CREAM-PUFs are robust (i.e., they repeatedly produce the same output), unique (i.e., they do not coincide with any other identically produced PUF), and unclonable (i.e., they are virtually impossible to replicate). Accordingly, CREAM-PUFs can serve as a foundational principle for establishing provenance attestation protocols for protecting intellectual property and confirming authenticity of engineered cell lines. Thus, disclosed herein are cells, nucleic acids, and methods for manufacturing, authenticating, and attesting the provenance of a cell line.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers, generally from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” or “hybridizes” refers to a process of establishing a non-covalent, sequence-specific interaction between two or more complementary strands of nucleic acids into a single hybrid, which in the case of two strands is referred to as a duplex.

The term “target” refers to a molecule that has an affinity for a given probe. Targets may be naturally-occurring or man-made molecules. Also, they can be employed in their unaltered state or as aggregates with other species.

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “about” as used herein when referring to a measurable value such as an amount, a percentage, and the like, is meant to encompass variations of ±20%, ±10%, ±5%, or ±1% from the measurable value. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “indel” or “indel mutation” as used herein refers to insertion or deletion of nucleic acid bases in the genome of a cell or in the nucleic acid sequence of interest.

The term “barcode” or “genetic barcode” as used herein, generally refers to a label, or identifier, that conveys or is capable of conveying information about a genetic sequence containing the barcode or a cell containing the barcode. A barcode can be used to identify a barcoded sequence, a barcoded cell, or barcoded sample. While barcodes can have a variety of different formats (for example, barcodes can include: polynucleotide barcodes; random nucleic acid and/or amino acid sequences; and synthetic nucleic acid and/or amino acid sequences), as used herein, a genetic barcode generally refers to a nucleic acid sequence. Barcodes can allow for identification and/or quantification of individual sequencing-reads.

### Cells, Nucleic Acids, and Compositions

In one aspect, disclosed herein is a genetically modified cell comprising:
a nucleic acid comprising
a genetic barcode; and
an insertion or deletion mutation (indel mutation);
wherein the genetic barcode is adjacent to the indel mutation.

In some embodiments, the genetic barcode comprises at least four or more nucleotides (for example, at least four or more nucleotides, at least five or more nucleotides, at least six or more nucleotides, at least seven or more nucleotides, at least eight or more nucleotides, at least nine or more nucleotides, or at least ten or more nucleotides. In some embodiments, the genetic barcode comprises a four nucleotide barcode. In some embodiments, the genetic barcode comprises a five nucleotide barcode. In some embodiments, the genetic barcode comprises a six nucleotide barcode. In some embodiments, the genetic barcode comprises a seven nucleotide barcode. In some embodiments, the genetic barcode comprises a eight nucleotide barcode. In some embodiments, the genetic barcode comprises a nine nucleotide barcode. In some embodiments, the genetic barcode comprises a ten nucleotide barcode.

In some embodiments, the genetic barcode is selected from a genetic barcode library having at least 10 distinct genetic barcodes, at least 20 distinct genetic barcodes, at least 50 distinct genetic barcodes, at least 100 distinct genetic barcodes, at least 200 distinct genetic barcodes, at least 300 distinct genetic barcodes, at least 400 distinct genetic barcodes, at least 500 distinct genetic barcodes, at least 600 distinct genetic barcodes, at least 700 distinct genetic barcodes, at least 800 distinct genetic barcodes, at least 900 distinct genetic barcodes, or least 1000 distinct genetic barcodes.

In some embodiments, the genetic barcode is selected from a genetic barcode library having less than 2000 distinct genetic barcodes, less than 1500 distinct genetic barcodes, less than 1000 distinct genetic barcodes, less than 900 distinct genetic barcodes, less than 800 distinct genetic barcodes, less than 700 distinct genetic barcodes, less than 600 distinct genetic barcodes, less than 500 distinct genetic barcodes, less than 400 distinct genetic barcodes, less than 300 distinct genetic barcodes, less than 200 distinct genetic barcodes, or less than 100 distinct genetic barcodes.

In some embodiments, the genetic barcode is integrated into a genome of the cell via homologous recombination. In some embodiments, the genetic barcode is integrated into the genome of the cell via CRISPR/SpCas9-mediated homologous recombination. In some embodiments, the genetic barcode is integrated into the genome of the cell via transcription activator-like effector-based nuclease (TALEN)-mediated homologous recombination. In some embodiments, the genetic barcode is integrated into the genome of the cell via zinc finger nuclease-mediated homologous recombination. In some embodiments, the genetic barcode is integrated into the genome of the cell via base editor-mediated homologous recombination. In yet other embodiments, the genetic barcode is integrated into the genome of the cell via transposon-based insertion methods.

In some embodiments, a genome editing enzyme is selected from a zinc finger nuclease (ZFN), a transcription activator-like effector-based nuclease (TALEN), or a clustered regularly interspaced short palindromic repeats (CRISPR) system nuclease. In some embodiments, the genome editing enzyme is Cas9, or a variant or homolog thereof. In some embodiments, the genome editing enzyme is Cpf1, or a variant or homolog thereof.

In some embodiments, the genetic barcode is adjacent to the indel mutation, for example, within about 20 nucleotides, within about 50 nucleotides, within about 100 nucleotides, within about 200 nucleotides, within about 300 nucleotides, within about 400 nucleotides, within about 500 nucleotides, within about 700 nucleotides, or within about 1000 nucleotides. The term “adjacent”, as used herein for the distance between the genetic barcode and the indel mutation, means that the genetic barcode and the indel mutation and located close enough to be amplified within the same PCR reaction (same amplicon) by the same set of PCR primers.

In some embodiments, two or more barcodes can be used combinatorially in a concatenated sequence. Combinatorial use of barcodes in concatenated barcodes can facilitate generation of a high number of barcodes. A concatenated barcode comprises sub-barcodes in a single polynucleotide wherein the sub-barcodes are disposed along the polynucleotide sufficiently close to an adjacent sub-barcode such that the concatenated barcode can be identified from a single amplicon formed from a PCR amplification reaction.

In some embodiments, the nucleic acid further comprises a promoter. In some embodiments, the nucleic acid further comprises a truncated human cytomegalovirus (CMV) promoter. In some embodiments, the genetic barcode is located immediately upstream of the promoter. In some embodiments, the promoter is a pol II promoter. In some embodiments, the promoter is a viral promoter. In some embodiments, the promoter is a heterologous promoter.

In some embodiments, the nucleic acid further comprises a reporter gene. In some embodiments, the indel mutation is located within the reporter gene. In some embodiments, the indel mutation is located within an open reading frame of the reporter gene. In some embodiments, the reporter gene is a fluorescent reporter gene. In some embodiments, the fluorescent reporter gene is mKate. In one embodiment, the fluorescent gene or protein comprises mCherry (mCh). In some embodiments, the fluorescent gene or protein comprises GFP. In some embodiments, the fluorescent gene or protein comprises YFP.

In some embodiments, the indel mutation is stochastically generated. In some embodiments, the indel mutation is generated by a non-homologous end joining repair mechanism. In some embodiments, the indel mutation is from 1 to 16 nucleotides in length.

In some embodiments, the indel mutation is an insertion mutation that is one or more nucleotides in length (for example, 1, 2, 3, 4, 5, 6, 7, 8, 9, 10, 11, 12, 13, 14, 15, 16, 17, 18, 19, 20, 25, 30, 35, 40, or more nucleotides are inserted). In some embodiments, the indel mutation is a deletion mutation that deletes one or more nucleotides (for example, 1, 2, 3, 4, 5, 6, 7, 8, 9, 10, 11, 12, 13, 14, 15, 16, 17, 18, 19, 20, 25, 30, 35, 40, or more nucleotides are deleted).

In some embodiments, the barcode is a randomly generated barcode. In some embodiments, the indel mutation is a randomly generated indel mutation.

In some embodiments, the nucleic acid further comprises a selection marker gene. In some embodiments, the selection marker gene is an antibiotic resistance gene or drug resistance gene. In some embodiments, the antibiotic resistance gene or drug resistance gene is a hygromycin resistance gene. In some embodiments, the antibiotic resistance gene or drug resistance gene is a selected from the group consisting of puromycin, neomycin, blastocidin, bleomycin, and hygromycin.

In some embodiments, the cell is a mammalian cell. In some embodiments, the cell is a human cell. In some embodiments, the cell is from a HEK293 cell line, an HCT116 cell line, or a HeLa cell line. In some embodiments, the genetic barcode is integrated into an AAVS1 locus of the HEK293 cell line. In some embodiments, the genetic barcode is integrated into a locus of the cell line that does not interfere with or alter the functioning of the cell. In some embodiments, the genetic barcode is integrated into other genomic locations, for example, CCR5, ROSA26, and H11.

In some embodiments, the cell is a eukaryotic cell. In some embodiments, the cell is a mouse cell. In some embodiments, the cell is a rat cell. In some embodiments, the cell is a yeast cell. In some embodiments, the cell is a prokaryotic cell.

In some embodiments, the cell, prior to genetic modification, does not comprise the genetic barcode and/or the indel mutation.

In another aspect, disclosed herein is a genetically modified nucleic acid, comprising:
a genetic barcode;
a promoter, wherein the promoter is operably linked to a reporter gene; and
an insertion or deletion mutation (indel mutation), wherein the indel mutation is located within the reporter gene.

In some embodiments, the genetic barcode comprises a five nucleotide barcode. In some embodiments, the genetic barcode is selected from a genetic barcode library having at least 100 distinct genetic barcodes. In some embodiments, the genetic barcode is integrated into a genome of the cell via homologous recombination. In some embodiments, the genetic barcode is integrated into the genome of the cell via CRISPR/SpCas9-mediated homologous recombination.

In some embodiments, the nucleic acid further comprises a promoter. In some embodiments, the nucleic acid further comprises a truncated human cytomegalovirus (CMV) promoter. In some embodiments, the genetic barcode is located immediately upstream of the promoter.

In some embodiments, the nucleic acid further comprises a reporter gene. In some embodiments, the indel mutation is located within the reporter gene. In some embodiments, the indel mutation is located within an open reading frame of the reporter gene. In some embodiments, the reporter gene is a fluorescent reporter gene. In some embodiments, the fluorescent reporter gene is mKate.

In some embodiments, the indel mutation is stochastically generated. In some embodiments, the indel mutation is generated by a non-homologous end joining repair mechanism. In some embodiments, the indel mutation is from 1 to 16 nucleotides in length.

In some embodiments, the nucleic acid further comprises a selection marker gene. In some embodiments, the selection marker gene is an antibiotic resistance gene. In some embodiments, the antibiotic resistance gene is a hygromycin resistance gene.

In some aspects, disclosed herein is a DNA vector comprising a nucleic acid as described herein. In some aspects, disclosed herein is a cell comprising a nucleic acid as described herein.

In some embodiments, the cell is a mammalian cell. In some embodiments, the cell is a human cell. In some embodiments, the cell is from a HEK293 cell line, an HCT116 cell line, or a HeLa cell line. In some embodiments, the genetic barcode is integrated into an AAVS1 locus of the HEK293 cell line.

In some embodiments, the nucleic acid is a heterologous nucleic acid. In some embodiments, the nucleic acid is a recombinant nucleic acid. In some embodiments, the nucleic acid is integrated into a genome of the cell. In some embodiments, the cell, prior to integration of the nucleic acid into the genome of the cell, does not comprise the genetic barcode and/or the indel mutation.

In some embodiments, the cell line comprises a population of genetically modified cells, comprising:
a plurality of genetic barcodes; and
a plurality of indel mutations;
wherein the plurality of genetic barcodes are adjacent to the plurality of indel mutations.

### Methods

In some aspects, disclosed herein is a method of manufacturing a cell line, comprising the steps of:
integrating a genetic barcode into a genome of a cell; and
integrating an insertion or deletion mutation (indel mutation) into the genome of the cell adjacent to the genetic barcode.

In some embodiments, the genetic barcode comprises at least four or more nucleotides (for example, at least four or more nucleotides, at least five or more nucleotides, at least six or more nucleotides, at least seven or more nucleotides, at least eight or more nucleotides, at least nine or more nucleotides, or at least ten or more nucleotides. In some embodiments, the genetic barcode comprises a four nucleotide barcode. In some embodiments, the genetic barcode comprises a five nucleotide barcode. In some embodiments, the genetic barcode comprises a six nucleotide barcode. In some embodiments, the genetic barcode comprises a seven nucleotide barcode. In some embodiments, the genetic barcode comprises a eight nucleotide barcode. In some embodiments, the genetic barcode comprises a nine nucleotide barcode. In some embodiments, the genetic barcode comprises a ten nucleotide barcode.

In some embodiments, the genetic barcode is selected from a genetic barcode library having at least 10 distinct genetic barcodes, at least 20 distinct genetic barcodes, at least 50 distinct genetic barcodes, at least 100 distinct genetic barcodes, at least 200 distinct genetic barcodes, at least 300 distinct genetic barcodes, at least 400 distinct genetic barcodes, at least 500 distinct genetic barcodes, at least 600 distinct genetic barcodes, at least 700 distinct genetic barcodes, at least 800 distinct genetic barcodes, at least 900 distinct genetic barcodes, or least 1000 distinct genetic barcodes.

In some embodiments, the genetic barcode is selected from a genetic barcode library having less than 2000 distinct genetic barcodes, less than 1500 distinct genetic barcodes, less than 1000 distinct genetic barcodes, less than 900 distinct genetic barcodes, less than 800 distinct genetic barcodes, less than 700 distinct genetic barcodes, less than 600 distinct genetic barcodes, less than 500 distinct genetic barcodes, less than 400 distinct genetic barcodes, less than 300 distinct genetic barcodes, less than 200 distinct genetic barcodes, or less than 100 distinct genetic barcodes.

In some embodiments, the genetic barcode is integrated into a genome of the cell via homologous recombination. In some embodiments, the genetic barcode is integrated into the genome of the cell via CRISPR/SpCas9-mediated homologous recombination. In some embodiments, the genetic barcode is integrated into the genome of the cell via transcription activator-like effector-based nuclease (TALEN)-mediated homologous recombination. In some embodiments, the genetic barcode is integrated into the genome of the cell via zinc finger nuclease-mediated homologous recombination. In some embodiments, the genetic barcode is integrated into the genome of the cell via base editor-mediated homologous recombination.

In some embodiments, a genome editing enzyme is selected from a zinc finger nuclease (ZFN), a transcription activator-like effector-based nuclease (TALEN), or a clustered regularly interspaced short palindromic repeats (CRISPR) system nuclease. In some embodiments, the genome editing enzyme is Cas9, or a variant or homolog thereof. In some embodiments, the genome editing enzyme is Cpf1, or a variant or homolog thereof.

In some embodiments, the genetic barcode is adjacent to the indel mutation, for example, within about 20 nucleotides, within about 50 nucleotides, within about 100 nucleotides, within about 200 nucleotides, within about 300 nucleotides, within about 400 nucleotides, within about 500 nucleotides, within about 700 nucleotides, or within about 1000 nucleotides. The term “adjacent”, as used herein for the distance between the genetic barcode and the indel mutation, means that the genetic barcode and the indel mutation and located close enough to be amplified within the same PCR reaction (same amplicon) by the same set of PCR primers.

In some embodiments, two or more barcodes can be used combinatorially in a concatenated sequence. Combinatorial use of barcodes in concatenated barcodes can facilitate generation of a high number of barcodes. A concatenated barcode comprises sub-barcodes in a single polynucleotide wherein the sub-barcodes are disposed along the polynucleotide sufficiently close to an adjacent sub-barcode such that the concatenated barcode can be identified from a single amplicon formed from a PCR amplification reaction.

In some embodiments, the nucleic acid further comprises a promoter. In some embodiments, the nucleic acid further comprises a truncated human cytomegalovirus (CMV) promoter. In some embodiments, the genetic barcode is located immediately upstream of the promoter. In some embodiments, the promoter is a pol II promoter. In some embodiments, the promoter is a viral promoter. In some embodiments, the promoter is a heterologous promoter.

In some embodiments, the nucleic acid further comprises a reporter gene. In some embodiments, the indel mutation is located within the reporter gene. In some embodiments, the indel mutation is located within an open reading frame of the reporter gene. In some embodiments, the reporter gene is a fluorescent reporter gene. In some embodiments, the fluorescent reporter gene is mKate. In one embodiment, the fluorescent gene or protein comprises mCherry (mCh). In some embodiments, the fluorescent gene or protein comprises GFP. In some embodiments, the fluorescent gene or protein comprises YFP.

In some embodiments, the indel mutation is stochastically generated. In some embodiments, the indel mutation is generated by a non-homologous end joining repair mechanism. In some embodiments, the indel mutation is from 1 to 16 nucleotides in length.

In some embodiments, the indel mutation is an insertion mutation that is one or more nucleotides in length (for example, 1, 2, 3, 4, 5, 6, 7, 8, 9, 10, 11, 12, 13, 14, 15, 16, 17, 18, 19, 20, 25, 30, 35, 40, or more nucleotides are inserted). In some embodiments, the indel mutation is a deletion mutation that deletes one or more nucleotides (for example, 1, 2, 3, 4, 5, 6, 7, 8, 9, 10, 11, 12, 13, 14, 15, 16, 17, 18, 19, 20, 25, 30, 35, 40 or more nucleotides are deleted).

In some embodiments, the barcode is a randomly generated barcode. In some embodiments, the indel mutation is a randomly generated indel mutation.

In some embodiments, the nucleic acid further comprises a selection marker gene. In some embodiments, the selection marker gene is an antibiotic resistance gene or drug resistance gene. In some embodiments, the antibiotic resistance gene or drug resistance gene is a hygromycin resistance gene. In some embodiments, the antibiotic resistance gene or drug resistance gene is a selected from the group consisting of puromycin, neomycin, blastocidin, bleomycin, and hygromycin.

In some embodiments, the cell is a mammalian cell. In some embodiments, the cell is a human cell. In some embodiments, the cell is from a HEK293 cell line, an HCT116 cell line, or a HeLa cell line. In some embodiments, the genetic barcode is integrated into an AAVS1 locus of the HEK293 cell line. In some embodiments, the genetic barcode is integrated into a locus of the cell line that does not interfere with or alter the functioning of the cell.

In some embodiments, the cell is a eukaryotic cell. In some embodiments, the cell is a mouse cell. In some embodiments, the cell is a rat cell. In some embodiments, the cell is a yeast cell. In some embodiments, the cell is a prokaryotic cell.

In some embodiments, the cell, prior to genetic modification, does not comprise the genetic barcode and/or the indel mutation.

In some embodiments, the indel mutation is generated by non-homologous end joining (NHEJ) repair. In some embodiments, the indel mutation is generated via CRISPR/SpCas9-mediated non-homologous end joining (NHEJ) repair. In some embodiments, a genome editing enzyme is selected from a zinc finger nuclease (ZFN), a transcription activator-like effector-based nuclease (TALEN), a clustered regularly interspaced short palindromic repeats (CRISPR) system nuclease, or a base editor.

In some aspects, disclosed herein is a method for authenticating a cell line, comprising the steps of:
generating a database defining a set of linked genetic barcodes and insertion or deletion mutations (indel mutations) from a reference cell line;
extracting sequence information from a target cell line defining a set of linked genetic barcodes and indel mutations from the target cell line;
comparing the set of linked genetic barcodes and indel mutations from the target cell line to the database defining the set of linked genetic barcodes and indel mutations from the reference cell line; and
determining a matching probability between the target cell line and the reference cell line in the database.

In some embodiments, the database defines a set of linked genetic barcodes and insertion or deletion mutations (indel mutations) from a number of different reference cell lines. In some embodiments, the matching probability is determined between the target cell line and any one of the different reference cell lines in the database, and a cell line is authenticated or validated if there are any matching probabilities below a set threshold. In some embodiments, this threshold is set through supervised machine learning models trained using the contents of the database. In some embodiments, fuzzy pattern matching methods are used to allow for a flexible threshold which can account for typical levels of sequencing errors.

In some embodiments, the genetic barcode comprises a five nucleotide barcode. In some embodiments, the genetic barcode is selected from a genetic barcode library having at least 100 distinct genetic barcodes. In some embodiments, the genetic barcode is integrated into a genome of the cell via homologous recombination. In some embodiments, the genetic barcode is integrated into the genome of the cell via CRISPR/SpCas9-mediated homologous recombination.

In some embodiments, the nucleic acid further comprises a promoter, wherein the promoter is operably linked to a reporter gene. In some embodiments, the nucleic acid further comprises a truncated human cytomegalovirus (CMV) promoter. In some embodiments, the genetic barcode is located immediately upstream of the promoter.

In some embodiments, the nucleic acid further comprises a reporter gene. In some embodiments, the indel mutation is located within the reporter gene. In some embodiments, the indel mutation is located within an open reading frame of the reporter gene. In some embodiments, the reporter gene is a fluorescent reporter gene. In some embodiments, the fluorescent reporter gene is mKate.

In some embodiments, the indel mutation is stochastically generated. In some embodiments, the indel mutation is generated by a non-homologous end joining repair mechanism. In some embodiments, the indel mutation is from 1 to 16 nucleotides in length.

In some embodiments, the nucleic acid further comprises a selection marker gene. In some embodiments, the selection marker gene is an antibiotic resistance gene. In some embodiments, the antibiotic resistance gene is a hygromycin resistance gene.

In some embodiments, the cell is a mammalian cell. In some embodiments, the cell is a human cell. In some embodiments, the cell is from a HEK293 cell line, an HCT116 cell line, or a HeLa cell line. In some embodiments, the genetic barcode is integrated into an AAVS1 locus of the HEK293 cell line.

In some embodiments, the cell, prior to genetic modification, does not comprise the genetic barcode and/or the indel mutation.

In some embodiments, the indel mutation is generated by non-homologous end joining (NHEJ) repair. In some embodiments, the indel mutation is generated via CRISPR/SpCas9-mediated non-homologous end joining (NHEJ) repair.

In some embodiments, the matching probability is determined using a Bray-Curtis dissimilarity analysis. In some embodiments, the matching probability is determined using total variation distance. In some embodiments, the matching probability is determined by any elementwise vector comparison metric.

In some embodiments, disclosed herein is a two-dimensional mapping library, comprising:
a first axis corresponding to one or more genetic barcodes integrated into a nucleic acid sequence of a cell line; and
a second axis corresponding to one or more indel mutations inserted into the nucleic acid sequence of the cell line.

In some embodiments, multiple CREAM-PUFs are integrated into a cell. In some embodiments, two or more CREAM-PUFs are integrated into a cell.

### EXAMPLES

The following examples are set forth below to illustrate the compounds, compositions, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Provenance Attestation of Cells Using Physical Unclonable Functions

Recent advances in synthetic biology and genome editing have enabled development of a broad range of engineered cells and have fueled emergence of a novel industry which seeks to produce specialized cell lines and monetize them through commercial distribution networks. Many such highly customized proprietary cell lines are the result of extensive and expensive research and development efforts and come with price tags in the tens of thousands of dollars. Therefore, the legitimate producers of these valuable cell lines have a vested interest to protect their intellectual property and recover their investment by ensuring that their proprietary cell line does not get illicitly copied and distributed. At the same time, customers who acquire such expensive cell lines also have a vested interest in being assured of the origin (and, thereby, the quality) of their purchase, as well as holding proof of legitimate ownership of the cell line. In short, this emerging industry is in need of novel protocols for formally verifying the sale transaction of proprietary cell lines.

Moreover, cross-contamination or misidentification of cell lines due to poor handling, mislabeling, or procurement from dubious or undocumented sources is a rampant problem, resulting in innumerable financial and time losses. For example, a major German cell repository has reported that 20% of its human cell line stocks were cross contaminated with other cell lines, and the China Center for Type Culture Collection demonstrated that 85% of cell lines in their repository, supposedly established from primary isolates, were actually HeLa cells. Such issues undermine quality, repeatability and, ultimately, overall efficiency of medical research. Therefore, quality control and source verification provisions are paramount toward safeguarding against working with unsuitable cell line models and producing false data.

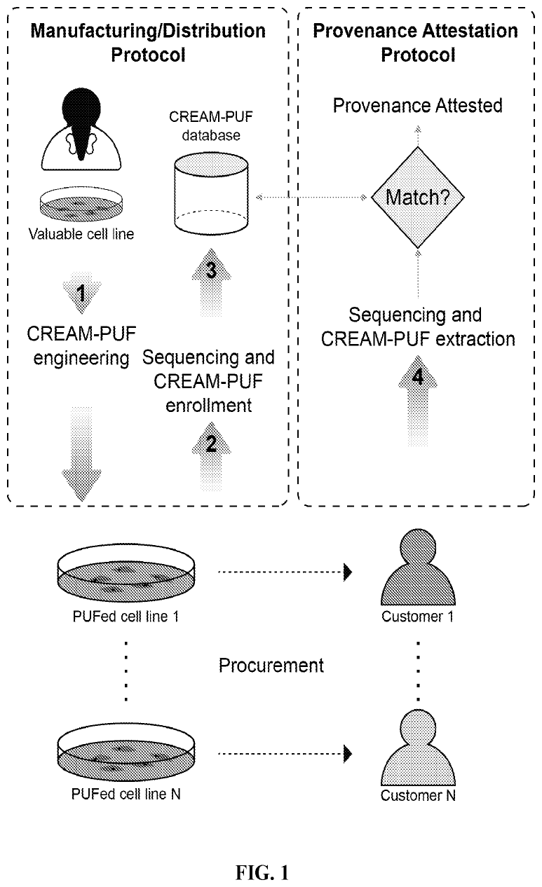

Disclosed herein is CRISPR Engineered Authentication of Mammalian Cells (CREAM-PUFs), a methodology which enables provenance attestation of cell lines through the use of the first genetic Physical Unclonable Functions (PUFs). A PUF is a hardware security primitive which exploits the inherent randomness of its manufacturing process to enable attestation of the entity wherein it is embodied. A PUF is typically modeled as a mapping between input stimuli (challenges) and output values (responses), which is established stochastically among a vast array of options and is, therefore, unique and irreproducible. Upon manufacturing, a PUF is interrogated and a database comprising valid Challenge-Response Pairs (CRPs) produced by this PUF is populated (FIG. 1). Attestation can, thus, be achieved by issuing a challenge to the holder of the physical entity embodying the PUF, receiving the response and comparing against the golden references stored in the database. Accordingly, typical quality metrics for evaluating a PUF include robustness, i.e., the probability that given the same challenge it will consistently produce the same response, and uniqueness, i.e., the probability that its mapping does not coincide with the mapping of any other identically manufactured PUF.

While PUF-like concepts were proposed earlier in the literature, their popularity soared after their first implementation in silicon, as part of electronic integrated circuits. Indeed, by exploiting the inherent variation of advanced semiconductor manufacturing processes, silicon PUFs became a commercial success, serving as the foundation of many security protocols implemented both in software and in hardware. While this success stimulated similar efforts in various other domains, to date PUFs have yet to be adopted in the context of biological sciences, wherein they could find numerous applications. Similar to the use of silicon PUFs (in their simplest form) as unique IDs for verifying genuineness of electronic circuits, genetic PUFs could be embedded in cell lines to attest their provenance.

More specifically, CREAM-PUFs could enable the producer of a valuable cell line to insert a unique, robust and unclonable signature in each legitimately produced copy of this cell line. Upon thawing of a frozen sample and prior to its initial use, a customer who purchased a copy of the cell line can obtain this signature and communicate it to the producer who compares it against the signature database of legitimately produced copies of this cell line and, thereby, attests its provenance (FIG. 1). Through this protocol, the producer of the cell line can ensure that anyone publicly claiming ownership of a copy of this cell line has acquired it legitimately. At the same time, the customer can be assured of the source and quality of the procured cell line, as the producer explicitly confirms its origin and assumes responsibility for its production.

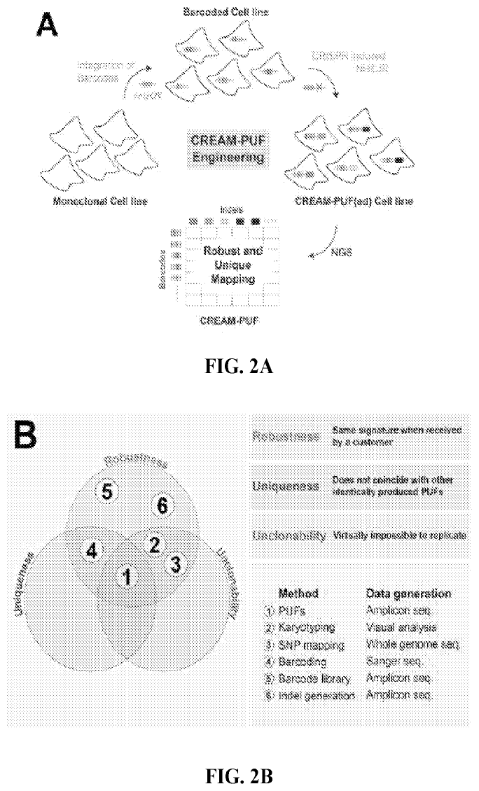

Toward developing CREAM-PUFs, it was hypothesized that a process which combines molecular barcoding with non-homologous end joining (NHEJ) repair and exploits the inherent stochasticity of the latter (FIG. 2A), yields measurable genetic changes that satisfy all PUF conditions. More specifically, a two-dimensional mapping between barcodes and indels resulting from this process, which can be obtained by sequencing a genetic locus of the cell line, is a robust yet unique and unclonable signature.

As visualized in a Venn diagram (FIG. 2B), CREAM-PUFs is the only methodology that satisfies all three PUF criteria of robustness, uniqueness, and unclonability. Robustness refers to the ability of a technology to produce the same signature when received by a customer. Uniqueness refers to the ability of a technology to not coincide with other identically produced PUFs. Unclonability refers to a technology that is virtually impossible to replicate. Barcodes and indels alone are not PUFs and cannot be used for provenance attestation. Indels are not PUFs because they are not unique and are clonable (thus violate two of the three PUF conditions). Barcodes are also not PUFs, as they violate the uniqueness criterion. Indeed, as shown later in this manuscript, when a 5-nucleotide barcode library was integrated into the AAVS1 locus of human HEK293 cells via CRISPR/SpCas9 in six parallel replicates, it was observed that the uniqueness criterion is not satisfied. Increasing the size of the barcode would not resolve the uniqueness criterion but would merely increase complexity. In contract, the uniqueness of the PUF design is not based on a scalar property, such as the complexity or entropy of barcodes or indels, but rather on the joint probability distributions of both barcodes and indels in the cell population. Finally, natural genetic variations such as short nucleotide polymorphisms (SNPs) or short tandem repeats (STRs) can indeed be used for cell line authentication but not for provenance attestation, because they are, generally, not unique or unclonable (FIG. 2B). As an example, all cell lines derived from a single monoclonal source share the same SNP mapping or karyotyping information and thus violate the uniqueness requirement. To implement the first generation of genetic PUFs, a pilot study was carried out where genome engineering using Clustered Regularly Interspaced Short Palindromic Repeats (CRISPR) was leveraged. CRISPR is an immune response mechanism against bacteriophage infections in bacteria and archaea that has revolutionized the field of genome editing and spurred myriads of applications critically relevant to agriculture, biomanufacturing, and human health. Critically, Cas9 can be programmed to bind to a specific region of DNA and generate a double stranded break which, in turn, initiates the error prone DNA repair pathway NHEJ. The method involved the following steps.

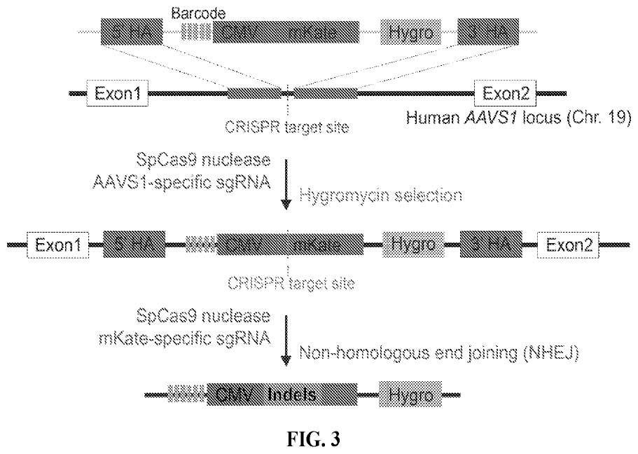

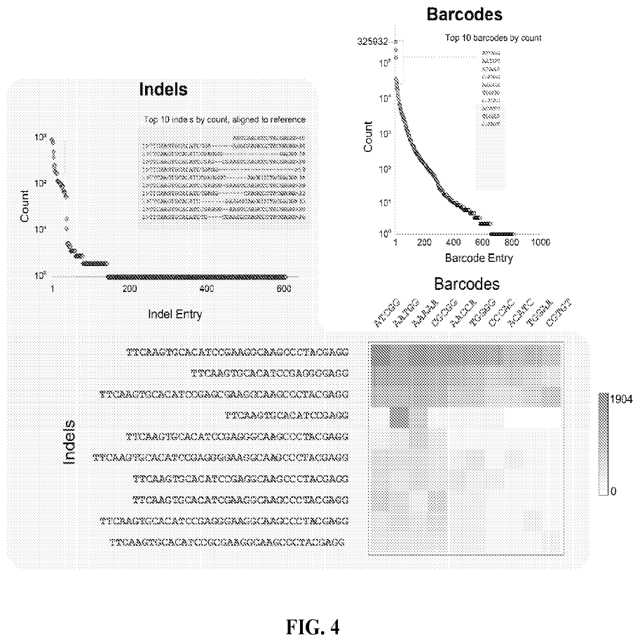

First, a 5-nucleotide barcode library was stably integrated into the AAVS1 locus of human HEK293 cells via CRISPR/SpCas9-mediated homologous recombination (HR). Specifically, as shown in FIG. 3, a 5-nucleotide barcode (5′-NNNNN-3′, complexity: 4⁵=1024) was placed immediately upstream of a truncated CMV (225 bp versus 612 bp of the full-length CMV promoter) mKate construct and a PGK1-hygromycin resistance gene for drug selection. The safe harbor AAVS1 locus was chosen as the integration site to minimize potential disruption of normal cellular functions upon the stable integration of the transgenes. Subsequently, the genomic DNA from the resulting stable cells was collected and used as the PCR template (Table 1, primers P1 and P2) to isolate the cDNA transcript harboring the barcode, which were subsequently subjected to NGS (Next Generation Sequencing)-based amplicon sequencing. In total, 805 distinct barcodes were detected (FIG. 4).

TABLE 1
Primer Sequences

| Primer | Sequence (5′→3′) |
|---|---|
| P1 | TCGTCGGCAGCGTCAGATGTGTATAAGAGACAGCCTTATATTCCCAGG |
| | GCCGGTTCGCGATCG (SEQ ID NO: 1) |
| P2 | GTCTCGTGGGCTCGGAGATGTGTATAAGAGACAGGCCGCCCTCGACCG |
| | CCTTGATTCTCATGGT (SEQ ID NO: 2) |
| P3 | CAGTACCCTGCAGGNNNNNTAGTTATTAATGACTCACGGGGATTTCCA |
| | AGTCTCCACCCC (SEQ ID NO: 3) |
| P4 | CAGTACGGCGCGCCCAATTTACGCGTTAAGATACATTGATGAGT (SEQ |
| | ID NO: 4) |
| P5 | CAGTACGGTACCAAGGTCGGGCAGGAAGAGGGCCTA (SEQ ID NO: 5) |
| P6 | CGGACTAGCCTTATTTTAACTTGCTATTTCTAGCTCTAAAACCCCTCGA |
| | CCGCCTTGATTCCGGTGTTTCGTCCTTTCCACAAGATA (SEQ ID NO: 6) |
| P7 | CAGTACTCTAGAAAAAAAGCACCGACTCGGTGCCACTTTTTCAAGTTG |
| | ATAACGGACTAGCCTTATTTTAACTTGC (SEQ ID NO: 7) |
| P8 | CGGACTAGCCTTATTTTAACTTGCTATTTCTAGCTCTAAAACCGGATGT |
| | GCACTTGAAGTGCGGTGTTTCGTCCTTTCCACAAGATA (SEQ ID NO: 8) |
| P9 | CGGACTAGCCTTATTTTAACTTGCTATTTCTAGCTCTAAAACTCGGATG |
| | TGCACTTGAAGTCGGTGTTTCGTCCTTTCCACAAGATA (SEQ ID NO: 9) |
| P10 | CGGACTAGCCTTATTTTAACTTGCTATTTCTAGCTCTAAAACTCGTAGG |
| | GCTTGCCTTCGCCGGTGTTTCGTCCTTTCCACAAGATA (SEQ ID NO: 10) |
| P11 | CGGACTAGCCTTATTTTAACTTGCTATTTCTAGCTCTAAAACTCGCCCT |
| | CGGATGTGCACTCGGTGTTTCGTCCTTTCCACAAGATA (SEQ ID NO: 11) |
| P12 | TCGTCGGCAGCGTCAGATGTGTATAAGAGACAG (SEQ ID NO: 12) |
| P13 | GTCTCGTGGGCTCGGAGATGTGTATAAGAGACAG (SEQ ID NO: 13) |

Next, it was aimed to combine the randomness of transfection into the barcoded cells and the inherent stochasticity of the cellular DNA error-repair processes to create a unique two-dimensional mapping between the barcodes and the indels. To this end, five sgRNAs (FIG. 12) were initially screened for targeting efficiency by designing the sgRNA to target the ORF of the fluorescence reporter mKate. As shown in FIG. 12, when co-transfected with SpCas9, all 5 sgRNAs efficiently suppressed the expression of mKate (sgRNA-5 was used for all subsequent experiments). We, therefore, proceeded by transiently transfecting the barcoded cell line with a sgRNA that targets adjacent to the integrated barcode in order to induce NHEJ repair.

Subsequently, the genomic DNA from the CRISPR-treated barcoded cell line was extracted and the amplicons containing both the barcodes and the indel sequences were prepared using PCR (primers P1 and P2). This was followed by NGS sequencing (100 bp paired-end reads), which provided both the barcode sequence (forward end) and the indel sequence (reverse end). As shown in FIG. 4, in total 569 distinct indels were observed and the most frequently occurring indels demonstrated deletions of 1- to 16-nucleotides flanking the predicted SpCas9 cutting site (cutting site: between 5′-CGAGGG-3′ and 5′-CGAAGG-3′, PAM: AGG).

The detected indels were associated with their corresponding barcodes from the same reads and the resulting two-dimensional matrix was sorted by the frequencies of barcoded indels. CRISPR-mediated editing occurred in a subpopulation of a non-uniformly distributed barcoded cell population, resulting in 218 out of the total 805 barcodes being present in the barcode and indels matrix. The cropped matrix is provided for the most frequently detected barcode and indel sequences in FIG. 4. By simple inspection, the utility of this matrix as a PUF to support CRispr-Engineered Authentication of Mammalian Cells (CREAM-PUF) becomes apparent: using silicon PUF terminology, a vector of (barcode, indel) elements in this matrix can be used as a challenge, while the corresponding vector of frequencies can be used as the response.

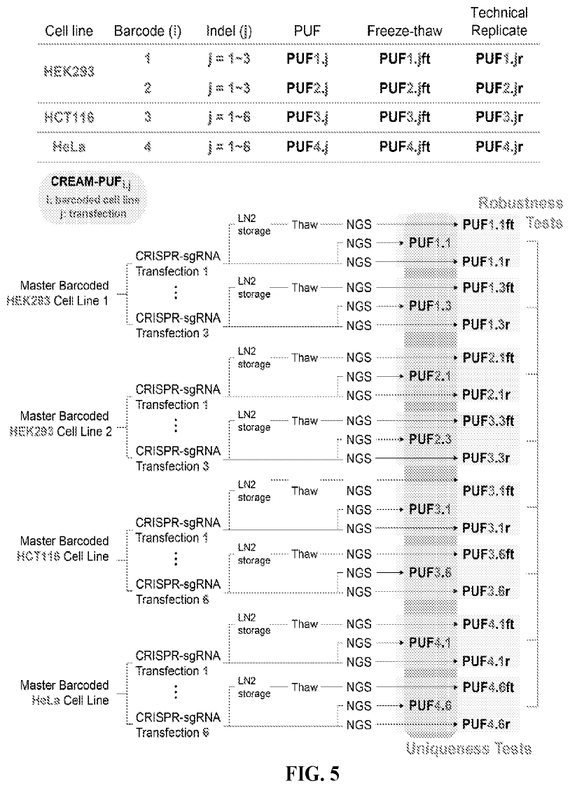

However, before relying on CREAM-PUFs for attesting provenance of a cell line, the aptitude as PUFs was evaluated (FIG. 2B). To this end, building upon the experience with the initial pilot experiments, CREAM-PUFs were thoroughly assessed using the strategy illustrated in FIG. 5. With numerous PUFs constructed across various human cell lines, individual PUF and pairwise comparisons were performed to establish their robustness (i.e., their ability to produce matching signatures when a cell line is sequenced multiple times, e.g., at the vendor and at the customer site) and uniqueness (i.e., their ability to produce distinct signatures when multiple, identically produced copies of the same cell line are sequenced).

To facilitate such comparisons, two independently engineered, barcoded cell lines (Barcoded Cell Line #1 and Barcoded Cell Line #2) were prepared for HEK293 cells. In parallel, two additional barcoded cell lines were also generated for HCT116 (Barcoded Cell Line #3) and HeLa (Barcodes Cell Line #4) cells, respectively. Next, for each of the two cell lines derived from HEK293, the barcoded cells were transfected with the same sgRNA (FIG. 12, sgRNA-5) three times (independent experiments), producing a total of 6 CREAM-PUFs (PUF1.1, PUF1.2 and PUF1.3 from Barcoded Cell Line #1, and PUF2.1, PUF2.2 and PUF2.3 from Barcoded Cell Line #2). All engineered cells were also subjected to one cycle of freezing and thawing, resulting in PUF1.1ft, PUF1.2ft, PUF1.3ft for Barcoded Cell Line #1 and PUF2.1ft, PUF2.2ft, PUF2.3ft for Barcoded Cell Line #2, respectively. These CREAM-PUFs were subjected to NGS analysis to produce the previously described barcode-indel matrix for each one of them. To incorporate and account for measurement errors introduced at the NGS step, PUF1.1 and PUF2.1 were sequenced twice, with the repeat results named PUF1.1r and PUF2.1r, respectively. Similarly, the two cell lines derived from HCT116 and HeLa were each subjected to 6 independent CRISPR/sgRNA treatments and the resulting cells (PUF3.j and PUF4.j, respectively) were subjected to one cycle of freezing and thawing (PUFi.jft, i={3,4}, j={1-6}), as well as repeated NGS sequencing (PUFi.jr, i={3,4}, j={1-6}). All the CREAM-PUFs produced by these experiments and used in the evaluation are summarized in FIG. 5.

To evaluate robustness, the NGS-generated barcode/indel matrix of PUFi.j was compared to those of PUFi.jr and PUFi.jft (i={1,2,3,4}), anticipating that they match (FIG. 5, Robustness Tests). Similarly, to evaluate CREAM-PUF uniqueness that stems from the stochastic nature of NHEJ repair and the random association with the barcodes, the NGS-generated barcode/indel matrix was compared across all PUFs (FIG. 5, Uniqueness Tests) anticipating that they are distinct.

For a qualitative assessment, the most densely populated area of the barcode/indel matrix were focused on. As an example, in FIG. 6A and FIG. 6B, the frequencies and sequences of the five most frequently observed barcodes and indels for PUFi.1, PUFi.1r and PUFi.1ft (i={1,2}, respectively) from HEK293 cells were provided. Also determined are heatmaps of the 30 most frequently observed barcodes and indels. These remain qualitatively the same and show a high level of robustness among these samples (which is be quantified in the following sections).

In contrast, different PUFs exhibit dissimilar patterns of the cropped CREAM-PUF matrices (e.g., PUF1.2 and PUF1.3 in FIG. 6C) and, importantly, different representation in the most frequently observed barcodes and indels. As an example, the 3^{rd} and 4^{th} most frequently observed barcodes for PUF1.2 were 5′-AATGG-3′ and 5′-AAAGC-3′, while for PUF1.3 they were 5′-AGGGA-3′ and 5′-AACCA-3′, respectively. Similarly, the most frequent indel from PUF1.2 was 5′-TTCAAGTGCACATCCGAGG-3′ (SEQ ID NO:14), while for PUF1.3 it was 5′-TTCAAGTGCACATCCGAAGGCAAGCCCTACGAGG-3′ (SEQ ID NO:15). These results show that a CREAM-PUF identifier based on a combination of the barcode/indel sequences and their respective counts can satisfy both robustness and uniqueness.

As mentioned earlier, 6 PUFs were introduced in each of two additional human cell lines (HCT116 and HeLa). The sequencing results (all PUFs in FIGS. 13-14) show that qualitatively these PUFs also satisfy both robustness and uniqueness. The frequencies and sequences of the five most frequently observed barcodes and indels for representative PUFs for both cell lines are provided (FIG. 7). For example, for HCT116 cells, the heatmaps were visually similar among PUF3.2, PUF3.2ft and PUF3.2r, while being distinct between PUF3.2 and the rest of the PUFs (FIG. 7A, FIG. 13). Similarly, for HeLa cells, while the 5^{th} most frequently observed indels from PUF4.2, PUF4.2ft and PUF4.2r remained as 5′-CCTCGGATGTGCACTTGAA-3′ (SEQ ID NO:16), this sequence was not observed in the most frequent indel list (top 5) from PUF4.1 sample (FIG. 7B, FIG. 14). All barcode and indel sequences from HCT116 and HeLa were determined.

For provenance attestation, the end-user of a CREAM-PUF(ed) cell line must provide the NGS data (i.e., barcode/indel matrix), which is then compared against the values stored in a database to determine whether there is a match. Importantly, to facilitate quantitative evaluation of the similarity between CREAM-PUF matrices, the barcode and indel sequences are first concatenated to generate unique addresses (FIG. 8). This allows expression of each CREAM-PUF as a probability distribution, based on the frequency of occurrence for each unique barcode-indel address.

To perform a pairwise comparison between CREAM-PUFs derived from each cell line, a standard metric is used for computing distance between probability distributions, the Total Variation Distance. The results (FIG. 9) reveal that intra-PUF distances (defined as the variation between a specific CREAM-PUFi.j and its corresponding repeat or freeze-thaw counterparts) are significantly smaller than inter-PUF distances (defined as the variation between two different CREAM-PUFs) in all three cell lines. As an example, in HEK293 cells, for each of the two PUFi families (i={1,2}), a threshold on Total Variation Distance can be selected (i.e., 0.007 and 0.019 respectively) such that all intra-PUF distances are below-threshold (indicating a match) and all inter-PUF distances are above-threshold (indicating a no-match). Similarly, such thresholds can also be established in PUFs derived from HCT116 and HeLa cells (0.037 for HCT116 and 0.013 for HeLa, respectively). This can also be visually confirmed by contrasting intra-PUF color intensity (i.e., inside the red boxes of FIG. 9) to inter-PUF color intensity (i.e., outside the red boxes) for each of the four PUFi families (i={1,2,3,4}).

In practice, provenance attestation can be performed quantitatively by using the Bray-Curtis dissimilarity between the end-user's CREAM-PUF and the values stored in a database. To demonstrate the use of the Bray-Curtis in this context, the intra-PUF and inter-PUF dissimilarities were computed using the rank-ordered N most-frequent barcode-indel addresses of PUF1.1 as the reference (FIG. 15A). As the number of used addresses increases towards the full list (N=3478), it was observed that the Bray-Curtis value between the reference (PUF1.1) and the CREAM-PUFs originating from the same Barcoded Cell Line #1 (i.e., PUF1.1r, PUF1.1ft, and PUF1.j where j={2,3}) also increases (FIG. 15B). On the other hand, the Bray-Curtis value from the CREAM-PUFs originating from Barcoded Cell Line #2 (i.e., PUF2.j where j={1,2,3}) remains close to the maximum (FIG. 15B). It was also observed that it is possible to obtain appreciably different intra-PUF and inter-PUF values by using as few as N=10 addresses (FIG. 15C). Indeed, it is unnecessary to use the complete list, since the contribution of additional barcode-indel addresses to the difference between intra-PUF and inter-PUF Bray-Curtis dissimilarities diminishes as N increases. Overall, it was observed that Bray-Curtis dissimilarity calculation using approximately 15% of the barcode-indel addresses (for all cell lines) results in lists that can provide an indisputable identification signature (FIGS. 15-20), while being sufficiently large to prevent unauthorized reproduction, as discussed later.

Based on the above observations, the Bray-Curtis dissimilarities were calculated between all the CREAM-PUFs in each of the three cell lines, each time using PUFi,j as a reference and comparing to its repeat and freeze-thaw versions, as well as to all other CREAM-PUFs. As shown therein, a Bray-Curtis distance of 0.2 is an appropriate threshold for matching a CREAM-PUF to its repeat and freeze-thaw counterparts in HEK293-derived PUFs (FIG. 10), while ensuring a no-match outcome when comparing to any other CREAM-PUF. For any given PUFs generated in a HEK293 cell line, the intra-PUF Bray-Curtis dissimilarity is never higher than 0.2, and the inter-PUF Bray-Curtis dissimilarities of these PUFs against those generated using the same set of barcodes (e.g., PUF1.2 vs PUF 1.3) was at least 2.6-fold higher than the corresponding intra-PUF Bray-Curtis dissimilarity. When compared against PUFs generated from a different set of barcodes (e.g., PUF1.2 vs PUF2.2), the difference rises to a minimum of 4.8-fold and a maximum of 12-fold increase in Bray-Curtis dissimilarity. It was observed that PUFs generated using HCT116 and HeLa cells show a similar trend (FIGS. 11A and 11B, respectively). As an example, using PUF3.1 as the reference, the inter-PUF Bray-Curtis dissimilarities were at least 3.4-fold higher than the corresponding intra-PUF dissimilarities (FIG. 11A and FIG. 21), a pattern which was even more pronounced in HeLa-derived PUF4.1 (>12-fold differences between inter- and intra-PUF dissimilarities, FIG. 11B and FIG. 22).

It is noted that a universal threshold is unnecessary, even if possible. In provenance attestation, it is sufficient to set an individual threshold for each cell line wherein a PUF has been introduced. Indeed, given a metric (e.g., Bray-Curtis dissimilarity), this threshold should be chosen to accept the signatures of all legitimately produced copies of the cell line, which the vendor stores in the CRP database, allowing a small margin to account for signature variation due to the freeze-thaw process or due to sequencing error, as further explained below. By individually setting this threshold for each cell line, its ability to differentiate between PUF signatures of legitimately produced copies and illegitimate clones of a cell line can be investigated and optimized.

In a noise-free case, the Bray-Curtis dissimilarity would be zero for valid PUFs. In reality, this is not the case. An important consideration here is that the Bray-Curtis values depend on the quality of the sequencing data. NGS is known to have a substitution error rate of 0.1-1% per base³⁹. Therefore, in addition to the repeated sequencing experiments (i.e., PUFi,jr) and to determine the worst-case Bray-Curtis dissimilarity values originating strictly from sequencing errors, for each of the reference PUFs derived from HEK293 cells 100 (artificially) mutated sequences were generated using an error rate of 1% per base. Subsequently, the Bray-Curtis values between these mutated sequences and their PUF references were calculated using the rank-ordered barcode-indel addresses of the reference. Using these simulations, the upper bound for the Bray-Curtis dissimilarity for “valid” PUFs was calculated (FIG. 23). The simulated worst-case dissimilarity values accurately match a CREAM-PUF to its repeat and freeze-thaw counterparts, while ensuring a no-match outcome when comparing to any other CREAM-PUF. The simulated worst-case dissimilarity values are different among PUF samples. This is because the underlying barcode distributions prior to applying the CRISPR-induced NHEJ are different and the absolute Bray-Curtis value depends on the average length of the sequencing reads (See Method Section, ‘Bray-Curtis and sequencing reads’).

As described earlier in FIG. 2B, barcodes alone do not satisfy the properties required to qualify as a PUF. To validate this claim, a 5-nucleotide barcode library was stably integrated into the AAVS1 locus of HEK293 cells in 6 parallel trials (BARCODE1-6), and subjected the samples to the two independent NGS-based amplicon sequencings. The overall barcode distribution patterns were strikingly similar among the repeats (FIG. 24A). Next, the Bray-Curtis dissimilarities between a BARCODEi and its sequencing repeat (BARCODEir), as well as between two distinct samples, were calculated as before (FIG. 24B). The intra-PUF dissimilarities generally overlapped with those of inter-PUFs (as an example, the Bray-Curtis dissimilarity between BARCODE2 and BARCODE2r was 0.013, which was higher than the Bray-Curtis dissimilarity between BARCODE3 and BARCODE4, which was 0.011). These results confirmed that barcodes alone do not satisfy the uniqueness requirement and therefore are not suitable to be used as PUFs.

To further investigate the uniqueness of the generated PUFs, additional computational analysis was performed. Specifically, it was tested whether the observed distribution of the barcode-indel addresses represents a unique combination of barcodes and indels that cannot be replicated. To achieve this, a barcode sequence and an indel sequence was randomly sampled from each of the reference HEK293-derived PUFs' probability distribution functions, and subsequently concatenated these two sequences to generate novel combinations of barcode-indel addresses (FIG. 25). The same number of concatenated addresses as in the original PUF was simulated to form a novel “resampled” PUF. Specifically, for each reference PUF, 100 resampled PUFs were generated. Next, the Bray-Curtis values between these simulated sequences and their PUF references were calculated. As shown in FIG. 26, for all reference CREAM-PUFs, the simulated inter-PUF dissimilarities (i.e., Bray-Curtis values between a reference and its reshuffled samples) are between 2.8× and 3.7× larger than intra-PUF dissimilarities (i.e., Bray-Curtis values between a reference and its repeat or freeze-thaw counterparts), and additionally, are all larger than the worst-case dissimilarity values identified in the earlier analysis.

Collectively, these additional computational and experimental results confirm that CREAM-PUFs satisfy both the robustness and the uniqueness criteria required for serving as a cell-line provenance attestation mechanism. It is further posited that CREAM-PUFs are also virtually impossible to replicate, thus unclonable. In the electronics industry, uniqueness and unclonability go hand-in-hand because silicon PUFs are inherent byproducts of the randomness of semiconductor manufacturing. Even if the PUF function is known, manufacturing an exact clone is impossible. In biology, counterfeiting a CREAM-PUF whose barcode-indel matrix is known would require DNA synthesis and integration of each individual sequence into a target cell line, followed by mixing the monoclonal cell populations to achieve the desired CREAM-PUF frequencies. While gene synthesis is becoming cheaper and synthesizing each individual fragment is feasible, integration, single cell isolation, mixing at desired proportions and, finally, validation requires prohibitive resource and time investment (See Method Section, ‘Reverse Engineering a CREAM-PUF’). Notably, the key determinants of synthesis costs and complexity (i.e., distance between the barcode and indel location and the number of barcode/indel combinations respectively) are dictated by the CREAM-PUF owner.

To summarize, a novel methodology is described herein that can be used to establish a provenance attestation protocol for commercial distribution of cell lines. Specifically, both the complexity of barcode libraries and the inherent stochasticity of DNA error-repair induced via genome editing was exploited to introduce physical unclonable functions in human cells. As valuable cell lines continue to emerge, provenance attestation to protect the investment and intellectual property of the producing company from illegal replication and to authenticate each clients' legitimate ownership of the purchased product is bound to become essential.

Prior to silicon PUFs, the lack of provenance attestation methods fueled a counterfeiting industry (IP theft through reverse engineering, illicit overproduction, IC recycling, remarking, etc.) resulting in an estimated annual loss of $100B by legitimate semiconductor companies. The invention of silicon PUFs has not only significantly curtailed the problem but has particularly succeeded in preventing counterfeiting of the latest cutting-edge products. Silicon PUFs were introduced for the purpose of providing a unique, robust, and unclonable digital fingerprint in each copy of a legitimately produced fabricated integrated circuit. While this digital fingerprint can be used as a key to support cryptographic algorithms, its main intent is provenance attestation of the integrated circuit.

Similarly, this methodology enables the producer of a valuable cell line to insert a unique, robust and unclonable signature in each legitimately produced copy of this cell line to support provenance attestation. Successful proliferation of such genetic PUFs can be transformative for intellectual property protection of engineered cell lines. Companies can introduce CREAM-PUFs to their cells to enable unique authorization and validation, labs across the world may use this technology as a starting point for validating point-of-source, and funding agencies and journals may require CREAM-PUFs in published documents and reports for quality control and for ensuring reproducibility.

### Methods

### Cell Culture and Transient Transfection

The HEK293 cells (catalog number: CRL-1573), HCT116 cells (catalog number: CCL-247), and HeLa cells (catalog number: CCL-2) were acquired from the American Type Culture Collection and maintained at 37° C., 100% humidity and 5% CO₂. The cells were grown in Dulbecco's modified Eagle's medium (DMEM, Invitrogen, catalog number: 11965-1181) supplemented with 10% Fetal Bovine Serum (FBS, Invitrogen, catalog number: 26140), 0.1 mM MEM non-essential amino acids (Invitrogen, catalog number: 11140-050), and 0.045 units/mL of Penicillin and 0.045 units/mL of Streptomycin (Penicillin-Streptomycin liquid, Invitrogen, catalog number: 15140). To pass the cells, the adherent culture was first washed with PBS (Dulbecco's Phosphate Buffered Saline, Mediatech, catalog number: 21-030-CM), then trypsinized with Trypsin-EDTA (0.25% Trypsin with EDTAX4Na, Invitrogen, catalog number: 25200) and finally diluted in fresh medium. For transient transfection, ˜300,000 cells in 1 mL of complete medium were plated into each well of 12-well culture treated plastic plates (Griener Bio-One, catalog number: 665180) and grown for 16-20 hours. All transfections were then performed using 1.75 μL of JetPRIME (Polyplus Transfection) and 75 μL of JetPRIME buffer. The transfection mixture was then applied to the cells and mixed with the medium by gentle shaking.

### Flow Cytometry

48-72 hours post transfection cells from each well of the 12-well plates were trypsinized with 0.1 mL 0.25% Trypsin-EDTA at 37° C. for 3 min. Trypsin-EDTA was then neutralized by adding 0.9 mL of complete medium. The cell suspension was centrifuged at 1,000 rpm for 5 min and after removal of supernatants, the cell pellets were re-suspended in 0.5 mL PBS buffer. The cells were analyzed on a BD LSRFortessa flow analyzer. CFP was measured with a 445-nm laser and a 515/20 band-pass filter, and mKate with a 561-nm laser, 610 emission filter and 610/20 band-pass filter. For data analysis, 100,000 events were collected. A FSC (forward scatter)/SSC (side scatter) gate was generated using a un-transfected negative sample and applied to all cell samples. The mKate and CFP readings from un-transfected HEK293 cells were set as baseline values and were subtracted from all other experimental samples. The normalized mKate values (mKate/CFP) were then collected and processed by FlowJo. All experiments were performed in triplicates.

### Generation of Barcoded Stable Cells

To generate the barcoded stable cells, ˜10 million of the cells were seeded onto a 10 cm petri dish. 16 hours later, the cells were transiently transfected with 1 μg of the donor plasmid (Barcode-Truncated CMV-mKate-PGK1-hygromycin resistance gene) and 9 μg of CMV-SpCas9-U6-AAVS1/sgRNA plasmid using the JetPRIME reagent (Polyplus Transfection). 48 hours later, hygromycin B (Thermo Fisher Scientific, catalog number: 10687010) was added at the final concentration of 200 μg/mL. The selection lasted ˜2 weeks, after which the surviving clones were pooled to generate the polyclonal stable cells. The barcoded stable cells were further expanded and maintained in the complete growth medium containing 200 μg/mL of hygromycin.

### Next Generation Sequencing (NGS)-Based Amplicon Sequencing

To determine the abundance of the barcode and indel sequences, total genomic DNA was isolated from CREAM-PUF cells transfected with CMV-SpCas9-U6-sgRNA5 using the DNeasy Blood & Tissue Kit (Qiagen, catalog number: 69504). cDNA fragments harboring both barcode and indel sequences were PCR amplified by using ˜100 ng of the genomic DNA and primers P1 and P2, which added the 5′-overhang adapter sequence P12 and the 3′-overhang adapter sequence P13 for subsequent Illumina NGS amplicon sequencing. The PCR conditions were: first one cycle of 30 s at 98° C., followed by 40 cycles of 10 s at 98° C., 30 s at 60° C., and 1 min at 72° C. The purified PCR products were then subjected to NGS-based amplicon sequencing (Illumina 100-bp paired end sequencing), which was performed at the Genome Sequencing Facility (GSF) at The University of Texas Health Science Center at San Antonio (UTHSCSA). 1 million individual reads were generated for each sample.

### Total Variation Distance

The total variation distance, δ_{TVD}, between two probability measures P and Q for a countable sample space Ω is equal to the half of the L¹ norm of these distributions or equivalently, half of the elementwise sum of the absolute difference of P and Q, as defined in Eq. 1.

$${δ}_{TVD} ( P , Q ) = \frac{1}{2} {‖ P - Q ‖}_{{L}_{1}} = \frac{1}{2} {\sum }_{ω ∈ Ω} \vert P ( ω ) - Q ( ω ) \vert \qquad Eq . 1$$

In addition, the total variation distance is the area between the two probability distribution curves defined as

$${δ}_{BC} ( u , v ) = \frac{{\sum }_{k = 1}^{n} \vert {u}_{k} - {v}_{k} \vert}{{\sum }_{k = 1}^{n} \vert {u}_{k} + {v}_{k} \vert} \qquad Eq . 2$$

It can be shown that for a finite set Ω, the total variation distance is equal to the largest difference in probability, taken over all subsets of Ω, i.e., all possible events.

### Bray-Curtis Dissimilarity

The Bray-Curtis dissimilarity δ_{BC} between two vectors u and v of same length n is defined in in Eq. 2:

$${δ}_{BC} ( u , v ) = \frac{{\sum }_{k = 1}^{n} \vert {u}_{k} - {v}_{k} \vert}{{\sum }_{k = 1}^{n} \vert {u}_{k} + {v}_{k} \vert} \qquad Eq . 2$$

The Bray-Curtis dissimilarity has values between zero and one when all coordinates are positive.

### General Cloning Protocols

Q5 High-Fidelity 2× Master Mix (New England Biolabs) was used for all polymerase chain reactions (PCR) according to the manufacturer's protocol. All oligonucleotides were ordered from Sigma-Aldrich and were listed in Table 1. The plasmids were constructed using PCR amplification, restriction digest (all restriction enzymes were ordered from New England Biolabs), and ligation with T4 DNA ligase (New England Biolabs). Gel purification and PCR purification were performed with QIAquick Gel Extraction and PCR Purification kits (Qiagen). Transformations were performed using NEB 5-alpha electrocompetent Escherichia coli (New England Biolabs). The minipreps were performed using QIAprep Spin Miniprep kit (Qiagen). The final plasmids were confirmed by both restriction enzyme digestions and direct Sanger sequencings.

### DNA Constructs

Barcode-Truncated CMV-mKate-PGK1-hygromycin resistance gene: CMV-mKate-PGK1-hygromycin resistance gene (unpublished results) was used as the PCR template with primers P3 and P4. The purified PCR product was then cloned into CMV-mKate-PGK1-hygromycin resistance gene vector using AscI and SbfI sites.

CMV-SpCas9-U6-sgRNA1: CMV-SpCas9-U6-BRIP1-sgRNA was used as the PCR template with primers P5 and P6. Next, the purified PCR product was used as the PCR template with primers P5 and P7. The purified PCR product was then cloned into CMV-SpCas9 (unpublished results) vector using KpnI and XbaI sites.

CMV-SpCas9-U6-sgRNA2: CMV-SpCas9-U6-BRIP1-sgRNA was used as the PCR template with primers P5 and P8. Next, the purified PCR product was used as the PCR template with primers P5 and P7. The purified PCR product was then cloned into CMV-SpCas9 (unpublished results) vector using KpnI and XbaI sites.

CMV-SpCas9-U6-sgRNA3: CMV-SpCas9-U6-BRIP1-sgRNA was used as the PCR template with primers P5 and P9. Next, the purified PCR product was used as the PCR template with primers P5 and P7. The purified PCR product was then cloned into CMV-SpCas9 (unpublished results) vector using KpnI and XbaI sites.

CMV-SpCas9-U6-sgRNA4: CMV-SpCas9-U6-BRIP1-sgRNA was used as the PCR template with primers P5 and P10. Next, the purified PCR product was used as the PCR template with primers P5 and P7. The purified PCR product was then cloned into CMV-SpCas9 (unpublished results) vector using KpnI and XbaI sites.

CMV-SpCas9-U6-sgRNA5: CMV-SpCas9-U6-BRIP1-sgRNA was used as the PCR template with primers P5 and P11. Next, the purified PCR product was used as the PCR template with primers P5 and P7. The purified PCR product was then cloned into CMV-SpCas9 (unpublished results) vector using KpnI and XbaI sites.

NGS (Next Generation Sequencing)-Based Amplicon Sequencing Data Analysis Pipeline with Sample Commands

Step 1: extracting the 100-bp reads

awk ‘NR%4==2’<f1.fastq|cat>f2.fastq
awk ‘NR%4==2’<r1.fastq|cat>r2.fastq

Step 2: joining the paired-end reads

paste -d ‘\0’ f2.fastq r2.fastq|cat>fr1.fastq

Step 3: filtering out corrupted reads

grep
“^{∧}ACTTATATTCCCAGGGCCGGTTCGCGATCGCCCTGCAGG[A-Z][A-Z][A-Z][A-Z][A-Z]
TAGTTATTAATGACTCACGGGGATTTCCAAGTCTCCACCCCATTGACGTCAATGGG
ACCGCCCTCGACCGCCTTGATTCTCATGGTCTGGGTGC[A-
Z]*GTGGTGGTTGTTCACGGTGCCCT”<fr1.fastq|cat>fr2.fastq

Step 4: extracting the barcode and indel sequences

sed-e
″s/CTTATATTCCCAGGGCCGGTTCGCGATCGCCCTGCAGG\(.*\)
TAGTTATTAATGACTCACGGGGATTTCCAAGTCTCCACCCCATTGACGTCAATGGGA
CCGCCCTCGACCGCCTTGATTCTCATGGTCTGGGTGC[A-
Z]*GTGGTGGTTGTTCACGGTGCCCT[A-Z]*∧1/″<fr2.fastq|cat>barcode1.fastq
sed-e
″s/CTTATATTCCCAGGGCCGGTTCGCGATCGCCCTGCAGG[A-Z][A-Z][A-Z][A-Z]A-Z]
TAGTTATTAATGACTCACGGGGATTTCCAAGTCTCCACCCCATTGACGTCAATGGG
ACCGCCCTCGACCGCCTTGATTCTCATGGTCTGGGTGC\(.*\)
GTGGTGGTTGTTCACGGTGCCCT[A-Z]*∧1/″<fr2.fastq|cat>indel1.fastq

Step 5: joining the paired barcode and indel sequences

paste -d ‘0’ barcode1.fastq indel1.fastq|cat>fr3.fastq

Step 6: isolating indels containing insertions/deletions

grep -v -x ‘.\{45}’ fr3.fastq|cat>fr4.fastq

### Reverse Engineering a CREAM-PUF

The effort needed to reverse engineer a CREAM-PUF, i.e., to synthesize a population that produces an identical barcode-indel matrix, requires an insurmountable amount of time, effort, and cost. Indeed, doing so would necessitate that each individual barcode/indel sequence pair be individually integrated into the required cell line, followed by monoclonal verification and, ultimately, mixing of the individual cells in the right proportions to reproduce the same barcode/indel frequencies observed from the CREAM-PUF. Simply installing the barcode/indel sequence can, on average, take a single researcher up to seven attempts over 19 weeks with 472 hours of hands-on time and approximately $18,000 to complete a single CRISPR editing workflow, i.e., generation of the desired monoclonal cell line. Furthermore, outsourcing a CRISPR-mediated genetic knock-in, such as a barcode/indel sequence described in our CREAM-PUFs, can have a starting price of $18,000-$25,000 with a similar time of completion. This process would simply produce cells with the same barcode/indel sequences contained in an individual CREAM-PUF. For example, to replicate PUF1.1, one would need to create 500 cell lines, which would cost at least $9 million. Moreover, to dial in the right frequency of engineered cells to reproduce the CREAM-PUF, would largely be trial and error with no guarantee that it is even possible.

### Bray-Curtis and Sequencing Reads

Assume that a PUF sample contains N barcode-indel reads, the average length of each read is L, and the error rate per base is e. Thus, the total number of mutations is N*L*e.

When N*L*e<<N, each mutation most likely will occur within a different read. It is further assumed that the mutation does not result in a sequence identical to one of the original reads. Thus, for the (N−N*L*e) non-mutated reads, they will appear in both the original and in the mutated samples. In contrast, for the (N*L*e) mutated reads, they will only appear in the original sample.

Therefore, the Bray-Curtis value will be: (N*L*e)/(N+N−N*L*e)=(L*e)/(2−L*e).

Since L*e<<1, the Bray-Curtis value is (L*e)/2, therefore the BC values are directly related to the read size L.

### REFERENCES

1. Rinaudo, K. et al. A universal RNAi-based logic evaluator that operates in mammalian cells. Nat. Biotechnol. 25, 795-801 (2007).
2. Moore, R. et al. CRISPR-based self-cleaving mechanism for controllable gene delivery in human cells. Nucleic Acids Res. 43, 1297-1303 (2015).
3. Weinberg, B. H. et al. Large-scale design of robust genetic circuits with multiple inputs and outputs for mammalian cells. Nat. Biotechnol. 35, 453-462 (2017).
4. Kim, T. & Lu, T. K. CRISPR/Cas-based devices for mammalian synthetic biology. Current Opinion in Chemical Biology 52, 23-30 (2019).
5. Chavez, A. et al. Highly efficient Cas9-mediated transcriptional programming. Nat. Methods 12, 326-328 (2015).
6. Cong, L. et al. Multiplex genome engineering using CRISPR/Cas systems. Science (80-.). 339, 819-823 (2013).
7. Leisner, M., Bleris, L., Lohmueller, J., Xie, Z. & Benenson, Y. Rationally designed logic integration of regulatory signals in mammalian cells. Nat. Nanotechnol. 5, 666-670 (2010).
8. Lapique, N. & Benenson, Y. Genetic programs can be compressed and autonomously decompressed in live cells. Nat. Nanotechnol. 13, 309-315 (2018).
9. Gao, X. J., Chong, L. S., Kim, M. S. & Elowitz, M. B. Programmable protein circuits in living cells. Science (80-.). 361, 1252 LP-1258 (2018).
10. Aijaz, A. et al. Biomanufacturing for clinically advanced cell therapies. Nat. Biomed. Eng. 2, 362-376 (2018).
11. Lee, J. S., Grav, L. M., Lewis, N. E. & Faustrup Kildegaard, H. CRISPR/Cas9-mediated genome engineering of CHO cell factories: Application and perspectives. Biotechnol. J. 10, 979-94 (2015).
12. Donohoue, P. D., Barrangou, R. & May, A. P. Advances in Industrial Biotechnology Using CRISPR-Cas Systems. Trends Biotechnol. 36, 134-146 (2018).
13. Quarton, T. et al. Uncoupling gene expression noise along the central dogma using genome engineered human cell lines. Nucleic Acids Res. 48, (2020).
14. Capes-Davis, A. et al. Check your cultures! A list of cross-contaminated or misidentified cell lines. International Journal of Cancer 127, 1-8 (2010).
15. MacLeod, R. A. F. et al. Widespread intraspecies cross-contamination of human tumor cell lines arising at source. Int. J. Cancer 83, 555-563 (1999).
16. Dirks, W. G. et al. Cell line cross-contamination initiative: An interactive reference database of STR profiles covering common cancer cell lines. International Journal of Cancer 126, 303-304 (2010).
17. Lichter, P. et al. Obligation for cell line authentication: Appeal for concerted action. International Journal of Cancer 126, 1 (2010).
18. Freshney, R. I. Database of misidentified cell lines. International Journal of Cancer 126, 302 (2010).
19. Cheung, S. T., Chan, S. L. & Lo, K. W. Contaminated and misidentified cell lines commonly use in cancer research. Mol. Carcinog. (2020). doi:10.1002/mc.23189
20. Rührmair, U., Sölter, J. & Sehnke, F. On the Foundations of Physical Unclonable Functions. Cryptol. ePrint Arch. 1-20 (2009).
21. Herder, C., Yu, M.-D., Koushanfar, F. & Devadas, S. Physical Unclonable Functions and Applications: A Tutorial. Proc. IEEE 102, 1126-1141 (2014).
22. McGrath, T., Bagci, I. E., Wang, Z. M., Roedig, U. & Young, R. J. A PUF taxonomy. Applied Physics Reviews 6, 011303 (2019).
23. Gao, Y., Al-Sarawi, S. F. & Abbott, D. Physical unclonable functions. Nature Electronics 3, 81-91 (2020).
24. Gassend, B., Clarke, D., van Dijk, M. & Devadas, S. Silicon physical random functions. in Proceedings of the 9th ACM conference on Computer and communications security—CCS '02 148-160 (ACM Press, 2002).
25. van Overbeek, M. et al. DNA Repair Profiling Reveals Nonrandom Outcomes at Cas9-Mediated Breaks. Mol. Cell 63, 633-646 (2016).
26. Chen, W. et al. Massively parallel profiling and predictive modeling of the outcomes of CRISPR/Cas9-mediated double-strand break repair. Nucleic Acids Res. 47, 7989-8003 (2019).
27. Shalem, O. et al. Genome-Scale CRISPR-Cas9 Knockout Screening in Human Cells. Science (80-.). 343, 84-87 (2014).
28. Jinek, M. et al. A programmable dual-RNA-guided DNA endonuclease in adaptive bacterial immunity. Science (80-.). 337, 816-821 (2012).
29. Mali, P. et al. RNA-guided human genome engineering via Cas9. Science (80-.). 339, 823-826 (2013).
30. Li, Y. Y., Nowak, C. M. C. M., Withers, D., Pertsemlidis, A. & Bleris, L. CRISPR-Based Editing Reveals Edge-Specific Effects in Biological Networks. Cris. J. 1, 286-293 (2018).
31. Hsu, P. D., Lander, E. S. & Zhang, F. Development and applications of CRISPR-Cas9 for genome engineering. Cell 157, 1262-1278 (2014).
32. Ran, F. A. et al. In vivo genome editing using Staphylococcus aureus Cas9. Nature 520, 186-91 (2015).
33. Gilbert, L. A. A. et al. CRISPR-Mediated Modular RNA-Guided Regulation of Transcription in Eukaryotes. Cell 154, 442-451 (2013).
34. Qi, L. S. et al. Repurposing CRISPR as an RNA-Guided Platform for Sequence-Specific Control of Gene Expression. Cell 152, 1173-1183 (2013).
35. Yang, L., Mali, P., Kim-Kiselak, C. & Church, G. CRISPR-Cas-mediated targeted genome editing in human cells. Gene Correct. 1114, 245-267 (2014).
36. Sadelain, M., Papapetrou, E. P. & Bushman, F. D. Safe harbours for the integration of new DNA in the human genome. Nat. Rev. Cancer 12, 51-58 (2012).
37. Nowak, C. M. C. M., Lawson, S., Zerez, M. & Bleris, L. Guide RNA engineering for versatile Cas9 functionality. Nucleic Acids Res. 44, gkw908 (2016).
38. Chen, W. et al. Massively parallel profiling and predictive modeling of the outcomes of CRISPR/Cas9-mediated double-strand break repair. Nucleic Acids Res. 47, 7989-8003 (2019).
39. Petrackova, A. et al. Standardization of Sequencing Coverage Depth in NGS: Recommendation for Detection of Clonal and Subclonal Mutations in Cancer Diagnostics. Front. Oncol. 9, (2019).
40. Guin, U. et al. Counterfeit Integrated Circuits: A Rising Threat in the Global Semiconductor Supply Chain. Proc. IEEE 102, 1207-1228 (2014).
41. Synthego. CRISPR Benchmark Report. (2019).
42. CRISPR gene Editing Services-Genscript. Available at: genscript.com/CRISPR-genome-edited-mammalian-cell-lines.html.
43. Custom CRISPR Cell Line Engineering Service|Canopy Bio. Available at: canopybiosciences.com/custom-cell-line-engineering-2/.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES

Barcode-Truncated CMV-mKate-PGK1-hygromycin resistance gene Sequence
(SEQ ID NO: 17)
TAGGGGTTCCGCGCACATTTCCCCGAAAAGTGCCACCTGGCCAGCTCCCATAGCTCA
GTCTGGTCTATCTGCCTGGCCCTGGCCATTGTCACTTTGCGCTGCCCTCCTCTCGCCC
CCGAGTGCCCTTGCTGTGCCGCCGGAACTCTGCCCTCTAACGCTGCCGTCTCTCTCCT
GAGTCCGGACCACTTTGAGCTCTACTGGCTTCTGCGCCGCCTCTGGCCCACTGTTTCC
CCTTCCCAGGCAGGTCCTGCTTTCTCTGACCTGCATTCTCTCCCCTGGGCCTGTGCCG
CTTTCTGTCTGCAGCTTGTGGCCTGGGTCACCTCTACGGCTGGCCCAGATCCTTCCCT
GCCGCCTCCTTCAGGTTCCGTCTTCCTCCACTCCCTCTTCCCCTTGCTCTCTGCTGTGT
TGCTGCCCAAGGATGCTCTTTCCGGAGCACTTCCTTCTCGGCGCTGCACCACGTGAT
GTCCTCTGAGCGGATCCTCCCCGTGTCTGGGTCCTCTCCGGGCATCTCTCCTCCCTCA
CCCAACCCCATGCCGTCTTCACTCGCTGGGTTCCCTTTTCCTTCTCCTTCTGGGGCCT
GTGCCATCTCTCGTTTCTTAGGATGGCCTTCTCCGACGGATGTCTCCCTTGCGTCCCG
CCTCCCCTTCTTGTAGGCCTGCATCATCACCGTTTTTCTGGACAACCCCAAAGTACCC
CGTCTCCCTGGCTTTAGCCACCTCTCCATCCTCTTGCTTTCTTTGCCTGGACACCCCG
TTCTCCTGTGGATTCGGGTCACCTCTCACTCCTTTCATTTGGGCAGCTCCCCTACCCC
CCTTACCTCTCTAGTCTGTGCTAGCTCTTCCAGCCCCCTGTCATGGCATCTTCCAGGG
GTCCGAGAGCTCAGCTAGTCTTCTTCCTCCAACCCGGGCCCCTATGTCCACTTCAGG
ACAGCATGTTTGCTGCCTCCAGGGATCCTGTGTCCCCGAGCTGGGACCACCTTATAT
TCCCAGGGCCGGTTCGCGATCGCCCTGCAGGNNNNNTAGTTATTAATGACTCACGGG
GATTTCCAAGTCTCCACCCCATTGACGTCAATGGGAGTTTGTTTTGGCACCAAAATC
AACGGGACTTTCCAAAATGTCGTAACAACTCCGCCCCATTGACGCAAATGGGCGGT
AGGCGTGTACGGTGGGAGGTCTATATAAGCAGAGCTGGTTTAGTGAACCGACCAGC
TAAGACACTGCCACGGTCAGATCCGCTAGCGCTACCGGTCGCCACCATGGTGAGCG
AGCTGATTAAGGAGAACATGCACATGAAGCTGTACATGGAGGGCACCGTGAACAAC
CACCACTTCAAGTGCACATCCGAGGGCGAAGGCAAGCCCTACGAGGGCACCCAGAC
CATGAGAATCAAGGCGGTCGAGGGCGGCCCTCTCCCCTTCGCCTTCGACATCCTGGC
TACCAGCTTCATGTACGGCAGCAAAACCTTCATCAACCACACCCAGGGCATCCCCGA
CTTCTTTAAGCAGTCCTTCCCCGAGGGCTTCACATGGGAGAGAGTCACCACATACGA
AGACGGGGGCGTGCTGACCGCTACCCAGGACACCAGCCTCCAGGACGGCTGCCTCA
TCTACAACGTCAAGATCAGAGGGGTGAACTTCCCATCCAACGGCCCTGTGATGCAG
AAGAAAACACTCGGCTGGGAGGCCTCCACCGAGACCCTGTACCCCGCTGACGGCGG
CCTGGAAGGCAGAGCCGACATGGCCCTGAAGCTCGTGGGCGGGGGCCACCTGATCT
GCAACTTGAAGACCACATACAGATCCAAGAAACCCGCTAAGAACCTCAAGATGCCC
GGCGTCTACTATGTGGACAGAAGACTGGAAAGAATCAAGGAGGCCGACAAAGAGA
CCTACGTCGAGCAGCACGAGGTGGCTGTGGCCAGATACTGCGACCTCCCTAGCAAA
CTGGGGCACAGAGGTGGAGGAGGTTCCGGATCTCACGGCTTCCCTCCCGAGGTGGA
GGAGCAGGCCGCCGGCACCCTGCCCATGAGCTGCGCCCAGGAGAGCGGCATGGATA
GACACCCTGCTGCTTGCGCCAGCGCCAGGATCAACGTCTCTAGATAACTGATCATAA
TCAGCCATACCACATTTGTAGAGGTTTTACTTGCTTTAAAAAACCTCCCACACCTCCC
CCTGAACCTGAAACATAAAATGAATGCAATTGTTGTTGTTAACTTGTTTATTGCAGC
TTATAATGGTTACAAATAAAGCAATAGCATCACAAATTTCACAAATAAAGCATTTTT
TTCACTGCATTCTAGTTGTGGTTTGTCCAAACTCATCAATGTATCTTAACGCGTAAAT
TGGGCGCGCCCTTAAGCTGGGACGGAGGCTTGTTTGCGAGGCCGCGGCCGGCCGAA
GTTCCTATTCTCTAGAAAGTATAGGAACTTCTACCGGGTAGGGGAGGCGCTTTTCCC
AAGGCAGTCTGGAGCATGCGCTTTAGCAGCCCCGCTGGGCACTTGGCGCTACACAA
GTGGCCTCTGGCCTCGCACACATTCCACATCCACCGGTAGGCGCCAACCGGCTCCGT
TCTTTGGTGGCCCCTTCGCGCCACCTTCTACTCCTCCCCTAGTCAGGAAGTTCCCCCC
CGCCCCGCAGCTCGCGTCGTGCAGGACGTGACAAATGGAAGTAGCACGTCTCACTA
GTCTCGTGCAGATGGACAGCACCGCTGAGCAATGGAAGCGGGTAGGCCTTTGGGGC
AGCGGCCAATAGCAGCTTTGCTCCTTCGCTTTCTGGGCTCAGAGGCTGGGAAGGGGT
GGGTCCGGGGGCGGGCTCAGGGGCGGGCTCAGGGGCGGGGCGGGCGCCCGAAGGT
CCTCCGGAGGCCCGGCATTCTGCACGCTTCAAAAGCGCACGTCTGCCGCGCTGTTCT
CCTCTTCCTCATCTCCGGGCCTTTCGACCTGCATCCATCTAGATCTCGATCGAGCAGC
TGAAGCTTACCGCAGGCTATGAAAAAGCCTGAACTCACCGCGACGTCTGTCGAGAA
GTTTCTGATCGAAAAGTTCGACAGCGTCTCCGACCTGATGCAGCTCTCGGAGGGCGA
AGAATCTCGTGCTTTCAGCTTCGATGTAGGAGGGCGTGGATATGTCCTGCGGGTAAA
TAGCTGCGCCGATGGTTTCTACAAAGATCGTTATGTTTATCGGCACTTTGCATCGGCC
GCGCTCCCGATTCCGGAAGTGCTTGACATTGGGGAATTCAGCGAGAGCCTGACCTAT
TGCATCTCCCGCCGTGCACAGGGTGTCACGTTGCAAGACCTGCCTGAAACCGAACTG
CCCGCTGTTCTGCAGCCGGTCGCGGAGGCCATGGATGCGATCGCTGCGGCCGATCTT
AGCCAGACGAGCGGGTTCGGCCCATTCGGACCGCAAGGAATCGGTCAATACACTAC
ATGGCGTGATTTCATATGCGCGATTGCTGATCCCCATGTGTATCACTGGCAAACTGT
GATGGACGACACCGTCAGTGCGTCCGTCGCGCAGGCTCTCGATGAGCTGATGCTTTG
GGCCGAGGACTGCCCCGAAGTCCGGCACCTCGTGCACGCGGATTTCGGCTCCAACA
ATGTCCTGACGGACAATGGCCGCATAACAGCGGTCATTGACTGGAGCGAGGCGATG
TTCGGGGATTCCCAATACGAGGTCGCCAACATCTTCTTCTGGAGGCCGTGGTTGGCT
TGTATGGAGCAGCAGACGCGCTACTTCGAGCGGAGGCATCCGGAGCTTGCAGGATC
GCCGCGGCTCCGGGCGTATATGCTCCGCATTGGTCTTGACCAACTCTATCAGAGCTT
GGTTGACGGCAATTTCGATGATGCAGCTTGGGCGCAGGGTCGATGCGACGCAATCG
TCCGATCCGGAGCCGGGACTGTCGGGCGTACACAAATCGCCCGCAGAAGCGCGGCC
GTCTGGACCGATGGCTGTGTAGAAGTACTCGCCGATAGTGGAAACCGACGCCCCAG
CACTCGTCCGAGGGCAAAGGAATAGGGGAGGCTAACTGAAGCTTCCCGGGGGTACC
AAATTCGTCGACAGATCTAACTTGTTTATTGCAGCTTATAATGGTTACAAATAAAGC
AATAGCATCACAAATTTCACAAATAAAGCATTTTTTTCACTGCATTCTAGTTGTGGTT
TGTCCAAACTCATCAATGTATCTTATGATGTCTGCATATGGAAGTTCCTATTCTCTAG
AAAGTATAGGAACTTCGCGGCCGCTCCCACCCGCTCGTCCCCCCGCGCACCTTTGCT
AGGAGCGGGTCGCCCATGTGGCTCTCAGGTTCTGGGTACTTTTATCTGTCCCCTCCAC
CCCACAGTGGGGCCACTAGGGACAGGATTGGTGACAGAAAAGCCCCATCCTTAGGC
CTCCTCCTTCCTAGTCTCCTGATATTGGGTCTAACCCCCACCTCCTGTTAGGCAGATT
CCTTATCTGGTGACACACCCCCATTTCCTGGAGCCATCTCTCTCCTTGCCAGAACCTC
TAAGGTTTGCTTACGATGGAGCCAGAGAGGATCCTGGGAGGGAGAGCTTGGCAGGG
GGTGGGAGGGAAGGGGGGGATGCGTGACCTGCCCGGTTCTCAGTGGCCACCCTGCG
CTACCCTCTCCCAGAACCTGAGCTGCTCTGACGCGGCCGTCTGGTGCGTTTCACTGA
TCCTGGTGCTGCAGCTTCCTTACACTTCCCAAGAGGAGAAGCAGTTTGGAAAAACAA
AATCAGAATAAGTTGGTCCTGAGTTCTAACTTTGGCTCTTCACCTTTCTAGTCCCCAA
TTTATATTGTTCCTCCGTGCGTCAGTTTTACCTGTGAGATAAGGCCAGTAGCCAGCCC
CGTCCTGGCAGGGCTGTGGTGAGGAGGGGGGTGTCCGTGTGGAAAACTCCCTTTGTG
AGAATGGTGCGTCCTAGGTGTTCACCAGGTCGTGGCCGCCTCTACTCCCTTTCTCTTT
CTCCATCCTTCTTTCCTTAAAGAGTCCCCAGTGCTATCTGGGACATATTCCTCCGCCC
AGAGCAGGGTCCCGCTTCCCTAAGGCCCTGCTCTGGGCTTCTGGGTTTGAGTCCTTG
GCAAGCCCAGGAGAGGCGCTCAGGCTTCCCTGTCCCCCTTCCTCGTCCACCATCTCA
TGCCCCTGGCTCTCCTGCCCCTTCCCTACAGGGGTTCCTGGCTCTGCTCTTCAGACTG
AGCCCCGTTCCCCTGCATCCCCGTTCCCCTGCATCCCCCTTCCCCTGCATCCCCCAGA
GGCCCCAGGCCACCTACTTGGCCTGGACCCCACGAGAGGCCACCCCAGCCCTGTCTA
CCAGGCTGCCTTTTGGGTGGATTCTCCTCCAACTGTGGGGTGACTGCTTGGCAAACT
CACCGGTACCCGGCCGCGACTCTAGATCATAATCAGCTCGAGCCTTAACAAGCTTCG
AAACGATATGGGCTGAATACAAAAACGATATGGGCTGAATACAAAAACGATATGGG
CTGAATACAAACCGCTTGAAGTCTTTAATTAAACCGCTTGAAGTCTTTAATTAAACC
GCTTGAAGTCTTTAATTAAAGGATCCACCGGATCTAGATAACTGATCATAATCGCGG
CCGCACTCCTCAGGTGCAGGCTGCCTATCAGAAGGTGGTGGCTGGTGTGGCCAATGC
CCTGGCTCACAAATACCACTGAGATCTTTTTCCCTCTGCCAAAAATTATGGGGACAT
CATGAAGCCCCTTGAGCATCTGACTTCTGGCTAATAAAGGAAATTTATTTTCATTGC
AATAGTGTGTTGGAATTTTTTGTGTCTCTCACTCGGAAGGACATATGGGAGGGCAAA
TCATTTAAAACATCAGAATGAGTATTTGGTTTAGAGTTTGGCAACATATGCCATATG
CTGGCTGCCATGAACAAAGGTGGCTATAAAGAGGTCATCAGTATATGAAACAGCCC
CCTGCTGTCCATTCCTTATTCCATAGAAAAGCCTTGACTTGAGGTTAGATTTTTTTTA
TATTTTGTTTTGTGTTATTTTTTTCTTTAACATCCCTAAAATTTTCCTTACATGTTTTAC
TAGCCAGATTTTTCCTCCTCTCCTGACTACTCCCAGTCATAGCTGTCCCTCTTCTCTTA
TGAAGATCCCTCGACCTGCAGCCCAAGCTTGGCGTAATCATGGTCATAGCTGTTTCC
TGTGTGAAATTGTTATCCGCTCACAATTCCACACAACATACGAGCCGGAAGCATAAA
GTGTAAAGCCTGGGGTGCCTAATGAGTGAGCTAACTCACATTAATTGCGTTGCGCTC
ACTGCCCGCTTTCCAGTCGGGAAACCTGTCGTGCCAGCGGATCCGCATCTCAATTAG
TCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGG
CCGCCTCGGCCTCTGAGCTATTCCAGAAGTAGTGAGGAGGCTTTTTTGGAGGCCTAG
GCTTTTGCAAAAAGCTAACTTGTTTATTGCAGCTTATAATGGTTACAAATAAAGCAA
TAGCATCACAAATTTCACAAATAAAGCATTTTTTTCACTGCATTCTAGTTGTGGTTTG
TCCAAACTCATCAATGTATCTTATCATGTCTGGATCCGCTGCATTAATGAATCGGCC
AACGCGCGGGGAGAGGCGGTTTGCGTATTGGGCGCTCTTCCGCTTCCTCGCTCACTG
ACTCGCTGCGCTCGGTCGTTCGGCTGCGGCGAGCGGTATCAGCTCACTCAAAGGCGG
TAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAG
CGTGGCGCTTTCTCAATGCTCACGCTGTAGGTATCTCAGTTCGGTGTAGGTCGTTCGC
TCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTT
GAAGTGGTGGCCTAACTACGGCTACACTAGAAGGACAGTATTTGGTATCTGCGCTCT
GCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGAGG
GCTTACCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTG
TCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCAT
AATTCTCTTACTGTCATGCCATCCGTAAGATGCTTTTCTGTGACTGGTGAGTACTCAA
CCAAGTCATTCTGAGAATAGTGTATGCGGCGACCGAGTTGCTCTTGCCCGGCGTCAA
TACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTG
GGTGAGCAAAAACAGGAAGGCAAAATGCCGCAAAAAAGGGAATAAGGGCGACACG
GAAATGTTGAATACTCATACTCTTCCTTTTTCAATATTATTGAAGCATTTATCAGGGT
TATTGTCTCATGAGCGGATACATATTTGAATGTATTTAGAAAAATAAACAAA
Left homology arm (SEQ ID NO: 18)
CCAGCTCCCATAGCTCAGTCTGGTCTATCTGCCTGGCCCTGGCCATTGTCACTTTGCG
CTGCCCTCCTCTCGCCCCCGAGTGCCCTTGCTGTGCCGCCGGAACTCTGCCCTCTAAC
GCTGCCGTCTCTCTCCTGAGTCCGGACCACTTTGAGCTCTACTGGCTTCTGCGCCGCC
TCTGGCCCACTGTTTCCCCTTCCCAGGCAGGTCCTGCTTTCTCTGACCTGCATTCTCT
CCCCTGGGCCTGTGCCGCTTTCTGTCTGCAGCTTGTGGCCTGGGTCACCTCTACGGCT
GGCCCAGATCCTTCCCTGCCGCCTCCTTCAGGTTCCGTCTTCCTCCACTCCCTCTTCC
CCTTGCTCTCTGCTGTGTTGCTGCCCAAGGATGCTCTTTCCGGAGCACTTCCTTCTCG
GCGCTGCACCACGTGATGTCCTCTGAGCGGATCCTCCCCGTGTCTGGGTCCTCTCCG
GGCATCTCTCCTCCCTCACCCAACCCCATGCCGTCTTCACTCGCTGGGTTCCCTTTTC
CTTCTCCTTCTGGGGCCTGTGCCATCTCTCGTTTCTTAGGATGGCCTTCTCCGACGGA
TGTCTCCCTTGCGTCCCGCCTCCCCTTCTTGTAGGCCTGCATCATCACCGTTTTTCTG
GACAACCCCAAAGTACCCCGTCTCCCTGGCTTTAGCCACCTCTCCATCCTCTTGCTTT
CTTTGCCTGGACACCCCGTTCTCCTGTGGATTCGGGTCACCTCTCACTCCTTTCATTT
GGGCAGCTCCCCTACCCCCCTTACCTCTCTAGTCTGTGCTAGCTCTTCCAGCCCCCTG
TCATGGCATCTTCCAGGGGTCCGAGAGCTCAGCTAGTCTTCTTCCTCCAACCCGGGC
CCCTATGTCCACTTCAGGACAGCATGTTTGCTGCCTCCAGGGATCCTGTGTCCCCGA
GCTGGGACCACCTTATATTCCCAGGGCCGGTT
5-nucleotide barcode
NNNNN
Truncated CMV promoter (SEQ ID NO: 19)
GACTCACGGGGATTTCCAAGTCTCCACCCCATTGACGTCAATGGGAGTTTGTTTTGG
CACCAAAATCAACGGGACTTTCCAAAATGTCGTAACAACTCCGCCCCATTGACGCA
AATGGGCGGTAGGCGTGTACGGTGGGAGGTCTATATAAGCAGAGCTGGTTTAGTGA
ACCGACCAGCTAAGACACTGCCACGGTCAGATCCGCTAGCGCTACCGGTCGCCACC
mKate open reading frame (SEQ ID NO: 20)
ATGGTGAGCGAGCTGATTAAGGAGAACATGCACATGAAGCTGTACATGGAGGGCAC
CGTGAACAACCACCACTTCAAGTGCACATCCGAGGGCGAAGGCAAGCCCTACGAGG
GCACCCAGACCATGAGAATCAAGGCGGTCGAGGGCGGCCCTCTCCCCTTCGCCTTCG
ACATCCTGGCTACCAGCTTCATGTACGGCAGCAAAACCTTCATCAACCACACCCAGG
GCATCCCCGACTTCTTTAAGCAGTCCTTCCCCGAGGGCTTCACATGGGAGAGAGTCA
CCACATACGAAGACGGGGGCGTGCTGACCGCTACCCAGGACACCAGCCTCCAGGAC
GGCTGCCTCATCTACAACGTCAAGATCAGAGGGGTGAACTTCCCATCCAACGGCCCT
GTGATGCAGAAGAAAACACTCGGCTGGGAGGCCTCCACCGAGACCCTGTACCCCGC
TGACGGCGGCCTGGAAGGCAGAGCCGACATGGCCCTGAAGCTCGTGGGCGGGGGCC
ACCTGATCTGCAACTTGAAGACCACATACAGATCCAAGAAACCCGCTAAGAACCTC
AAGATGCCCGGCGTCTACTATGTGGACAGAAGACTGGAAAGAATCAAGGAGGCCGA
CAAAGAGACCTACGTCGAGCAGCACGAGGTGGCTGTGGCCAGATACTGCGACCTCC
CTAGCAAACTGGGGCACAGA
PGK1 promoter (SEQ ID NO: 21)
CTGGGACGGAGGCTTGTTTGCGAGGCCGCGGCCGGCCGAAGTTCCTATTCTCTAGAA
AGTATAGGAACTTCTACCGGGTAGGGGAGGCGCTTTTCCCAAGGCAGTCTGGAGCA
TGCGCTTTAGCAGCCCCGCTGGGCACTTGGCGCTACACAAGTGGCCTCTGGCCTCGC
ACACATTCCACATCCACCGGTAGGCGCCAACCGGCTCCGTTCTTTGGTGGCCCCTTC
GCGCCACCTTCTACTCCTCCCCTAGTCAGGAAGTTCCCCCCCGCCCCGCAGCTCGCG
TCGTGCAGGACGTGACAAATGGAAGTAGCACGTCTCACTAGTCTCGTGCAGATGGA
CAGCACCGCTGAGCAATGGAAGCGGGTAGGCCTTTGGGGCAGCGGCCAATAGCAGC
TTTGCTCCTTCGCTTTCTGGGCTCAGAGGCTGGGAAGGGGTGGGTCCGGGGGCGGGC
TCAGGGGCGGGCTCAGGGGCGGGGCGGGCGCCCGAAGGTCCTCCGGAGGCCCGGC
ATTCTGCACGCTTCAAAAGCGCACGTCTGCCGCGCTGTTCTCCTCTTCCTCATCTCCG
GGCCTTTCGACCTGCATCCATCTAGATCTCGATCGAGCAGCTGAAGCTTACCGCAGG
CT
Hygromycin resistance gene open reading frame (SEQ ID NO: 22)
ATGAAAAAGCCTGAACTCACCGCGACGTCTGTCGAGAAGTTTCTGATCGAAAAGTTC
GACAGCGTCTCCGACCTGATGCAGCTCTCGGAGGGCGAAGAATCTCGTGCTTTCAGC
TTCGATGTAGGAGGGCGTGGATATGTCCTGCGGGTAAATAGCTGCGCCGATGGTTTC
TACAAAGATCGTTATGTTTATCGGCACTTTGCATCGGCCGCGCTCCCGATTCCGGAA
GTGCTTGACATTGGGGAATTCAGCGAGAGCCTGACCTATTGCATCTCCCGCCGTGCA
CAGGGTGTCACGTTGCAAGACCTGCCTGAAACCGAACTGCCCGCTGTTCTGCAGCCG
GTCGCGGAGGCCATGGATGCGATCGCTGCGGCCGATCTTAGCCAGACGAGCGGGTT
CGGCCCATTCGGACCGCAAGGAATCGGTCAATACACTACATGGCGTGATTTCATATG
CGCGATTGCTGATCCCCATGTGTATCACTGGCAAACTGTGATGGACGACACCGTCAG
TGCGTCCGTCGCGCAGGCTCTCGATGAGCTGATGCTTTGGGCCGAGGACTGCCCCGA
AGTCCGGCACCTCGTGCACGCGGATTTCGGCTCCAACAATGTCCTGACGGACAATGG
CCGCATAACAGCGGTCATTGACTGGAGCGAGGCGATGTTCGGGGATTCCCAATACG
AGGTCGCCAACATCTTCTTCTGGAGGCCGTGGTTGGCTTGTATGGAGCAGCAGACGC
GCTACTTCGAGCGGAGGCATCCGGAGCTTGCAGGATCGCCGCGGCTCCGGGCGTAT
ATGCTCCGCATTGGTCTTGACCAACTCTATCAGAGCTTGGTTGACGGCAATTTCGAT
GATGCAGCTTGGGCGCAGGGTCGATGCGACGCAATCGTCCGATCCGGAGCCGGGAC
TGTCGGGCGTACACAAATCGCCCGCAGAAGCGCGGCCGTCTGGACCGATGGCTGTG
TAGAAGTACTCGCCGATAGTGGAAACCGACGCCCCAGCACTCGTCCGAGGGCAAAG
GAA
Right homology arm (SEQ ID NO: 23)
GGTTCTGGGTACTTTTATCTGTCCCCTCCACCCCACAGTGGGGCCACTAGGGACAGG
ATTGGTGACAGAAAAGCCCCATCCTTAGGCCTCCTCCTTCCTAGTCTCCTGATATTGG
GTCTAACCCCCACCTCCTGTTAGGCAGATTCCTTATCTGGTGACACACCCCCATTTCC
TGGAGCCATCTCTCTCCTTGCCAGAACCTCTAAGGTTTGCTTACGATGGAGCCAGAG
AGGATCCTGGGAGGGAGAGCTTGGCAGGGGGTGGGAGGGAAGGGGGGGATGCGTG
ACCTGCCCGGTTCTCAGTGGCCACCCTGCGCTACCCTCTCCCAGAACCTGAGCTGCT
CTGACGCGGCCGTCTGGTGCGTTTCACTGATCCTGGTGCTGCAGCTTCCTTACACTTC
CCAAGAGGAGAAGCAGTTTGGAAAAACAAAATCAGAATAAGTTGGTCCTGAGTTCT
AACTTTGGCTCTTCACCTTTCTAGTCCCCAATTTATATTGTTCCTCCGTGCGTCAGTTT
TACCTGTGAGATAAGGCCAGTAGCCAGCCCCGTCCTGGCAGGGCTGTGGTGAGGAG
GGGGGTGTCCGTGTGGAAAACTCCCTTTGTGAGAATGGTGCGTCCTAGGTGTTCACC
AGGTCGTGGCCGCCTCTACTCCCTTTCTCTTTCTCCATCCTTCTTTCCTTAAAGAGTCC
CCAGTGCTATCTGGGACATATTCCTCCGCCCAGAGCAGGGTCCCGCTTCCCTAAGGC
CCTGCTCTGGGCTTCTGGGTTTGAGTCCTTGGCAAGCCCAGGAGAGGCGCTCAGGCT
TCCCTGTCCCCCTTCCTCGTCCACCATCTCATGCCCCTGGCTCTCCTGCCCCTTCCCTA
CAGGGGTTCCTGGCTCTGCTCTTCAGACTGAGCCCCGTTCCCCTGCATCCCCGTTCCC
CTGCATCCCCCTTCCCCTGCATCCCCCAGAGGCCCCAGGCCACCTACTTGGCCTGGA
CCCCACGAGAGGCCACCCCAGCCCTGTCTACCAGGCTGCCTTTTGGGTGGATTCTCC
TCCAACTGTGGGGTGACTGCTTGGCAAACTCAC

We claim:

1. A genetically modified cell comprising:
a nucleic acid comprising
a genetic barcode; and
an insertion or deletion mutation (indel mutation);
wherein the genetic barcode is adjacent to the indel mutation.

2. The cell of claim 1, wherein the genetic barcode comprises a five nucleotide barcode.

3. The cell of claim 1 or 2, wherein the genetic barcode is selected from a genetic barcode library having at least 100 distinct genetic barcodes.

4. The cell of any one of claims 1 to 3, wherein the genetic barcode is integrated into a genome of the cell via homologous recombination.

5. The cell of any one of claims 1 to 4, wherein the genetic barcode is integrated into the genome of the cell via CRISPR/SpCas9-mediated homologous recombination.

6. The cell of any one of claims 1 to 5, wherein the nucleic acid further comprises a promoter.

7. The cell of any one of claims 1 to 6, wherein the nucleic acid further comprises a truncated human cytomegalovirus (CMV) promoter.

8. The cell of claim 5 or 6, wherein the genetic barcode is located immediately upstream of the promoter.

9. The cell of any one of claims 1 to 8, wherein the nucleic acid further comprises a reporter gene.

10. The cell of claim 9, wherein the indel mutation is located within the reporter gene.

11. The cell of claim 9, wherein the indel mutation is located within an open reading frame of the reporter gene.

12. The cell of any one of claims 9 to 11, wherein the reporter gene is a fluorescent reporter gene.

13. The cell of claim 12, wherein the fluorescent reporter gene is mKate.

14. The cell of any one of claims 1 to 13, wherein the indel mutation is stochastically generated.

15. The cell of any one of claims 1 to 14, wherein the indel mutation is generated by a non-homologous end joining repair mechanism.

16. The cell of any one of claims 1 to 15, wherein the indel mutation is from 1 to 16 nucleotides in length.

17. The cell of any one of claims 1 to 16, wherein the nucleic acid further comprises a selection marker gene.

18. The cell of claim 17, wherein the selection marker gene is an antibiotic resistance gene.

19. The cell of claim 18, wherein the antibiotic resistance gene is a hygromycin resistance gene.

20. The cell of any one of claims 1 to 19, wherein the cell is a mammalian cell.

21. The cell of any one of claims 1 to 20, wherein the cell is a human cell.

22. The cell of any one of claims 1 to 21, wherein the cell is from a HEK293 cell line, an HCT116 cell line, or a HeLa cell line.

23. The cell of claim 22, wherein the genetic barcode is integrated into an AAVS1 locus of the HEK293 cell line.

24. The cell of any one of claims 1 to 23, wherein the cell, prior to genetic modification, does not comprise the genetic barcode and/or the indel mutation.

25. A genetically modified nucleic acid, comprising:
a genetic barcode;
a promoter, wherein the promoter is operably linked to a reporter gene; and
an insertion or deletion mutation (indel mutation), wherein the indel mutation is located within the reporter gene.

26. The nucleic acid of claim 25, wherein the genetic barcode comprises a five nucleotide barcode.

27. The nucleic acid of claim 25 or 26, wherein the genetic barcode is selected from a genetic barcode library having at least 100 distinct genetic barcodes.

28. The nucleic acid of any one of claims 25 to 27, wherein the genetic barcode is integrated into a genome of a cell via homologous recombination.

29. The nucleic acid of any one of claims 25 to 28, wherein the genetic barcode is integrated into the genome of the cell via CRISPR/SpCas9-mediated homologous recombination.

30. The nucleic acid of any one of claims 25 to 29, wherein the promoter comprises a human cytomegalovirus (CMV) promoter.

31. The nucleic acid of any one of claims 25 to 30, wherein the genetic barcode is located immediately upstream of the promoter.

32. The nucleic acid of any one of claims 25 to 31, wherein the indel mutation is located within an open reading frame of the reporter gene.

33. The nucleic acid of any one of claims 25 to 32, wherein the reporter gene is a fluorescent reporter gene.

34. The nucleic acid of claim 33, wherein the fluorescent reporter gene is mKate.

35. The nucleic acid of any one of claims 25 to 34, wherein the indel mutation is stochastically generated.

36. The nucleic acid of any one of claims 25 to 35, wherein the indel mutation is generated by a non-homologous end joining repair mechanism.

37. The nucleic acid of any one of claims 25 to 36, wherein the indel mutation is from 1 to 16 nucleotides in length.

38. The nucleic acid of any one of claims 25 to 37, wherein the nucleic acid further comprises a selection marker gene.

39. The nucleic acid of claim 38, wherein the selection marker gene is an antibiotic resistance gene.

40. The nucleic acid of claim 39, wherein the antibiotic resistance gene is a hygromycin resistance gene.

41. A DNA vector comprising the nucleic acid of any one of claims 25 to 40.

42. A cell comprising the nucleic acid of any one of claims 25 to 40.

43. The cell of claim 42, wherein the cell is a mammalian cell.

44. The cell of claim 42, wherein the cell is a human cell.

45. The cell of claim 42, wherein the cell is from a HEK293 cell line, an HCT116 cell line, or a HeLa cell line.

46. The cell of any one of claims 42 to 45, wherein the genetic barcode is integrated into an AAVS1 locus of the HEK293 cell line.

47. The cell of any one of claims 42 to 46, wherein the nucleic acid is integrated into a genome of the cell.

48. The cell of any one of claims 42 to 47, wherein the cell, prior to integration of the nucleic acid into the genome of the cell, does not comprise the genetic barcode and/or the indel mutation.

49. A method of manufacturing a cell line, comprising the steps of:
integrating a genetic barcode into a genome of a cell; and
integrating an insertion or deletion mutation (indel mutation) into the genome of the cell adjacent to the genetic barcode.

50. The method of claim 49, wherein the genetic barcode comprises a five nucleotide barcode.

51. The method of claim 49 or 50, wherein the genetic barcode is selected from a genetic barcode library having at least 100 distinct genetic barcodes.

52. The method of any one of claims 49 to 51, wherein the genetic barcode is integrated into a genome of the cell via homologous recombination.

53. The method of any one of claims 49 to 52, wherein the genetic barcode is integrated into the genome of the cell via CRISPR/SpCas9-mediated homologous recombination.

54. The method of any one of claims 49 to 53, wherein the cell further comprises a promoter, wherein the promoter is operably linked to a reporter gene.

55. The method of any one of claims 49 to 54, wherein the cell further comprises a truncated human cytomegalovirus (CMV) promoter.

56. The method of any one of claims 49 to 55, wherein the genetic barcode is located immediately upstream of the promoter.

57. The method of any one of claims 54 to 56, wherein the indel mutation is located within the reporter gene.

58. The method of any one of claims 54 to 57, wherein the indel mutation is located within an open reading frame of the reporter gene.

59. The method of any one of claims 54 to 58, wherein the reporter gene is a fluorescent reporter gene.

60. The method of claim 59, wherein the fluorescent reporter gene is mKate.

61. The method of any one of claims 49 to 60, wherein the indel mutation is stochastically generated.

62. The method of any one of claims 49 to 61, wherein the indel mutation is generated by a non-homologous end joining repair mechanism.

63. The method of any one of claims 49 to 62, wherein the indel mutation is from 1 to 16 nucleotides in length.

64. The method of any one of claims 49 to 63, wherein the cell further comprises a selection marker gene.

65. The method of claim 64, wherein the selection marker gene is an antibiotic resistance gene.

66. The method of claim 65, wherein the antibiotic resistance gene is a hygromycin resistance gene.

67. The method of any one of claims 49 to 66, wherein the cell is a mammalian cell.

68. The method of any one of claims 49 to 67, wherein the cell is a human cell.

69. The method of any one of claims 49 to 68, wherein the cell is from a HEK293 cell line, an HCT116 cell line, or a HeLa cell line.

70. The method of claim 69, wherein the genetic barcode is integrated into an AAVS1 locus of the HEK293 cell line.

71. The method of any one of claims 49 to 70, wherein the cell, prior to genetic modification, does not comprise the genetic barcode and/or the indel mutation.

72. The method of any one of claims 49 to 71, wherein the indel mutation is generated by non-homologous end joining (NHEJ) repair.

73. The method of any one of claims 49 to 72, wherein the indel mutation is generated via CRISPR/SpCas9-mediated non-homologous end joining (NHEJ) repair.

74. A method for authenticating a cell line, comprising the steps of:
generating a database defining a set of linked genetic barcodes and insertion or deletion mutations (indel mutations) from a reference cell line;
extracting sequence information from a target cell line defining a set of linked genetic barcodes and indel mutations from the target cell line;
comparing the set of linked genetic barcodes and indel mutations from the target cell line to the database defining the set of linked genetic barcodes and indel mutations from the reference cell line; and
determining a matching probability between the target cell line and the reference cell line in the database.

75. The method of claim 74, wherein the genetic barcodes comprise a five nucleotide barcode.

76. The method of claim 74 or 75, wherein the genetic barcodes are selected from a genetic barcode library having at least 100 distinct genetic barcodes.

77. The method of any one of claims 74 to 76, wherein the genetic barcodes are integrated into a genome of the target cell line via homologous recombination.

78. The method of any one of claims 74 to 77, wherein the genetic barcodes are integrated into the genome of the target cell line via CRISPR/SpCas9-mediated homologous recombination.

79. The method of any one of claims 74 to 78, wherein the target cell line further comprises a promoter, wherein the promoter is operably linked to a reporter gene.

80. The method of any one of claims 74 to 79, wherein the target cell line further comprises a truncated human cytomegalovirus (CMV) promoter.

81. The method of any one of claims 79 to 80, wherein the genetic barcodes are located immediately upstream of the promoter.

82. The method of any one of claims 74 to 81, wherein the indel mutation is located within the reporter gene.

83. The method of any one of claims 74 to 82, wherein the indel mutation is located within an open reading frame of the reporter gene.

84. The method of any one of claims 74 to 83, wherein the reporter gene is a fluorescent reporter gene.

85. The method of claim 84, wherein the fluorescent reporter gene is mKate.

86. The method of any one of claims 74 to 85, wherein the indel mutation is stochastically generated.

87. The method of any one of claims 74 to 86, wherein the indel mutation is generated by a non-homologous end joining repair mechanism.

88. The method of any one of claims 74 to 87, wherein the indel mutation is from 1 to 16 nucleotides in length.

89. The method of any one of claims 74 to 88, wherein the cell further comprises a selection marker gene.

90. The method of claim 89, wherein the selection marker gene is an antibiotic resistance gene.

91. The method of claim 90, wherein the antibiotic resistance gene is a hygromycin resistance gene.

92. The method of any one of claims 74 to 91, wherein the target cell line is a mammalian cell line.

93. The method of any one of claims 74 to 92, wherein the target cell line is a human cell line.

94. The method of any one of claims 74 to 93, wherein the target cell line is from a HEK293 cell line, an HCT116 cell line, or a HeLa cell line.

95. The method of claim 94, wherein the genetic barcode is integrated into an AAVS1 locus of the HEK293 cell line.

96. The method of any one of claims 74 to 95, wherein the target cell line, prior to genetic modification, does not comprise the genetic barcode and/or the indel mutation.

97. The method of any one of claims 74 to 96, wherein the indel mutation is generated by non-homologous end joining (NHEJ) repair.

98. The method of any one of claims 74 to 97, wherein the indel mutation is generated via CRISPR/SpCas9-mediated non-homologous end joining (NHEJ) repair.

99. The method of any one of claims 74 to 98, wherein the matching probability is determined using a Bray-Curtis dissimilarity analysis.
